(19) United States
(12) Patent Application Publication
(10) Pub. No.: US 2026/0096855 A1
(43) Pub. Date: Apr. 9, 2026

(54) METHOD AND DEVICE FOR DRIVING SURGICAL INSTRUMENT

(71) Applicant: LIVSMED INC., Seongnam-si (KR)

(72) Inventors: Young Jae SONG, Seongnam-si (KR); Jin Hyuk YOON, Seongnam-si (KR)

(21) Appl. No.: 19/056,346

(22) Filed: Feb. 18, 2025

(30) Foreign Application Priority Data
Oct. 4, 2024 (KR) 10-2024-0134850

(51) Int. Cl. A61B 34/20; A61B 34/37

(52) U.S. Cl. CPC A61B 34/20; A61B 34/37

(57) ABSTRACT

Provided are a method and device for driving a surgical instrument. The method may include generating first position information about a relationship between a position of a first robot and a position of a second robot, based on the position of the first robot. In addition, the method may further include generating second position information about a relationship between the position of the second robot and the position of the first robot, based on the position of the second robot. In addition, the method may further include generating third position information about a relationship between the position of the first robot and the position of the second robot, based on the first position information and the second position information.

### CROSS-REFERENCE TO RELATED APPLICATION

This application is based on and claims priority under 35 U.S.C. § 119 to Korean Patent Application No. 10-2024-0134850, filed on Oct. 4, 2024, in the Korean Intellectual Property Office, the disclosure of which is incorporated by reference herein in its entirety.

### BACKGROUND

### 1. Field

The present disclosure relates to a method and device for driving a surgical instrument.

### 2. Description of the Related Art

In medical terms, surgery refers to the treatment of diseases by cutting, incising, or manipulating a skin, a mucous membrane, or other tissues by using medical instruments. In particular, open surgery for incising and opening the skin of a surgical site to treat, shape, or remove an organ or the like therein causes issues such as bleeding, side effects, patient's pain, or scarring. Therefore, recently, surgery performed by forming a certain hole on a skin and inserting only a medical device, for example, a laparoscopic instrument or a surgical instrument, or surgery using a robot has been spotlighted as an alternative.

Here, a surgical robot refers to a robot that has a function of replacing a surgical action performed by a surgeon. Advantageously, the surgical robot may operate more accurately and precisely as compared with a human and enable remote surgery.

Meanwhile, a surgical robot is generally composed of a master robot and a slave robot. When a surgical operator manipulates a control lever (e.g., a handle) provided on the master robot, a surgical tool coupled to or held by a robotic arm on the slave robot is manipulated to perform surgery.

When controlling a plurality of slave robots through a master robot, determining the relative angles between the slave robots is an important factor. For example, it is necessary to determine the relative angles between a plurality of robotic arms to ensure the normal progress of a surgery.

Thus, in a system using a plurality of robotic arms included in a plurality of slave robots, there is a need for technology for determining the relative angles between the plurality of slave robots.

The above-mentioned background art is technical information possessed by the inventor for the derivation of the present disclosure or acquired during the derivation of the present disclosure, and cannot necessarily be said to be a known technique disclosed to the general public prior to the filing of the present disclosure.

### SUMMARY

Provided are a method and device for driving a surgical instrument. In addition, provided is a computer-readable recording medium having recorded thereon a program for causing a computer to execute the method.

Technical objectives of the present disclosure are not limited to the foregoing, and other unmentioned objectives or advantages of the present disclosure would be understood from the following description and be more clearly understood from the embodiments of the present disclosure. In addition, it would be appreciated that the objectives and advantages of the present disclosure may be implemented by means provided in the claims and a combination thereof.

According to a first aspect of the present disclosure, a method includes generating first position information about a relationship between a position of a first robot and a position of a second robot, based on the position of the first robot, generating second position information about a relationship between the position of the second robot and the position of the first robot, based on the position of the second robot, and generating third position information about a relationship between the position of the first robot and the position of the second robot, based on the first position information and the second position information.

According to a second aspect of the present disclosure, a device includes at least one memory, and at least one processor configured to generate first position information about a relationship between a position of a first robot and a position of a second robot, based on the position of the first robot, generate second position information about a relationship between the position of the second robot and the position of the first robot, based on the position of the second robot, and generate third position information about a relationship between the position of the first robot and the position of the second robot, based on the first position information and the second position information.

According to a third aspect of the present disclosure, a computer-readable recording medium may have recorded thereon a program for causing a computer to execute the method according to the first aspect.

In addition, other methods and systems for implementing the present disclosure, and a computer-readable recording medium having recorded thereon a computer program for executing the methods may be further provided.

Other aspects, features, advantages other than those described above will become apparent from the following drawings, claims, and detailed description of the present disclosure.

### BRIEF DESCRIPTION OF THE DRAWINGS

These and/or other aspects will become apparent and more readily appreciated from the following description of the embodiments, taken in conjunction with the accompanying drawings in which:

FIG. 1 is a diagram for describing an example of a system for driving a surgical instrument, according to an embodiment;

FIG. 2A is a configuration diagram illustrating an example of a user terminal according to an embodiment;

FIG. 2B is a configuration diagram illustrating an example of a server according to an embodiment;

FIG. 3 is a diagram for describing another example of a system for driving a surgical instrument, according to an embodiment;

FIG. 4 is a block diagram illustrating an internal configuration of a surgical robotic system of FIG. 3;

FIG. 5 is a perspective view illustrating a slave robot of the surgical robotic system of FIG. 3 and multi-joint surgical instruments mounted on the slave robot;

FIG. 6 is a perspective view illustrating a multi-joint surgical instrument according to an embodiment of the present disclosure;

FIGS. 7 and 8 are perspective views of an end tool of the multi-joint surgical instrument of FIG. 6;

FIG. 9 is a plan view of the end tool of the multi-joint surgical instrument of FIG. 6;

FIGS. 10 and 11 are perspective views of a driving part of the multi-joint surgical instrument of FIG. 6;

FIG. 12 is a plan view of the driving part of the multi-joint surgical instrument of FIG. 6;

FIG. 13 is a rear view of the driving part of the multi-joint surgical instrument of FIG. 6;

FIG. 14 is a side view of the driving part of the multi-joint surgical instrument of FIG. 6;

FIG. 15 is a diagram illustrating in detail components associated with a first jaw, among components including pulleys and wires of the multi-joint surgical instrument illustrated in FIG. 6;

FIG. 16 is a diagram illustrating in detail components associated with a second jaw, among components including pulleys and wires of the multi-joint surgical instrument illustrated in FIG. 6;

FIGS. 17A to 18C are diagrams illustrating a pitch motion of the multi-joint surgical instrument illustrated in FIG. 6;

FIGS. 19A to 20B are diagrams illustrating a yaw motion of the multi-joint surgical instrument illustrated in FIG. 6;

FIG. 21 is a flowchart for describing an example of a method of driving a surgical instrument, according to an embodiment;

FIGS. 22A and 22B are diagrams for describing examples of a first robot and a second robot, according to an embodiment;

FIG. 23 is a diagram for describing examples of a first sensor part and a second sensor part mounted respectively on the first robot and the second robot illustrated in FIGS. 22A and 22B;

FIG. 24 is a diagram for describing an example in which a processor generates fifth position information, according to an embodiment; and

FIG. 25 is a diagram for describing an example of a surgical instrument that operates based on driving information, according to an embodiment.

### DETAILED DESCRIPTION

Hereinafter, various embodiments of the present disclosure will be described with reference to the accompanying drawings. Various embodiments of the present disclosure may be variously modified and may have various embodiments, and particular embodiments are illustrated in the drawings and detailed descriptions related to the embodiments are described. However, this is not intended to limit various embodiments of the present disclosure to particular modes of practice, and it is to be appreciated that all changes, equivalents, and/or substitutes that do not depart from the spirit and technical scope of various embodiments of the present disclosure are encompassed in the present disclosure. With regard to the description of the drawings, similar reference numerals are used to refer to similar elements.

As used in various embodiments of the present disclosure, the expressions “include”, “may include”, and other conjugates refer to the existence of a corresponding disclosed function, operation, or constituent element, and do not limit one or more additional functions, operations, or constituent elements. In addition, as used in various embodiments of the present disclosure, the terms “include”, “have”, and other conjugates are intended merely to denote a certain feature, numeral, step, operation, element, component, or a combination thereof, and should not be construed to initially exclude the existence of or a possibility of addition of one or more other features, numerals, steps, operations, elements, components, or combinations thereof.

As used in various embodiments of the present disclosure, expressions such as “or” include any and all combinations of the listed words. For example, “A or B” may include A, may include B, or may include both A and B. In addition, as used herein, expressions such as “A or B”, “at least one of A or/and B”, or “one or more of A or/and B” may include all possible combinations of the listed items. For example, “A or B”, “at least one of A and B”, or “one or more of A or B” may refer to (1) at least one A, (2) at least one B, or (3) both at least one A and at least one B.

As used in various embodiments of the present disclosure, expressions such as “first” or “second” may modify various components of various embodiments, but do not limit the components. For example, the expressions do not limit the order and/or importance of the components. The expressions may be used to distinguish one component from another. For example, a first user device and a second user device are all user devices, and indicate different user devices. For example, a first element may be referred to as a second element, and a second element may be referred to as a first element in a similar manner, without departing from the scope of various embodiments of the present disclosure.

As used in embodiments of the present disclosure, terms such as “module”, “unit”, “part”, etc., denote a unit of a component that performs at least one function or operation, and may be implemented as hardware or software or a combination of hardware and software. In addition, a plurality of “modules”, “units”, “parts”, etc. may be integrated into at least one module or chip to be implemented as at least one processor, except for cases in which each of them needs to be implemented as separate particular hardware.

As used herein, the expression “configured to” may be interchangeably used with, for example, “suitable for”, “having the capacity to”, “designed to”, “adapted to”, “made to”, or “capable of”, according to a situation. The expression “configured to” may not imply only “specially designed to” in a hardware manner.

The terms used in various embodiments of the present disclosure are used only to describe a particular embodiment, and are not intended to limit the various embodiments of the present disclosure. The singular expression also includes the plural meaning as long as it is not inconsistent with the context.

Unless otherwise defined, all terms, including technical and scientific terms, used herein have the same meaning as commonly understood by those of skill in the art to which the present disclosure pertains based on an understanding of the present disclosure.

Terms, such as those defined in commonly used dictionaries, are to be interpreted as having a meaning that is consistent with their meaning in the context of the relevant art and various embodiments of the present disclosure, and are not to be interpreted in an idealized or overly formal sense unless expressly so defined herein.

Hereinafter, various embodiments of the present disclosure will be described in detail with reference to the accompanying drawings.

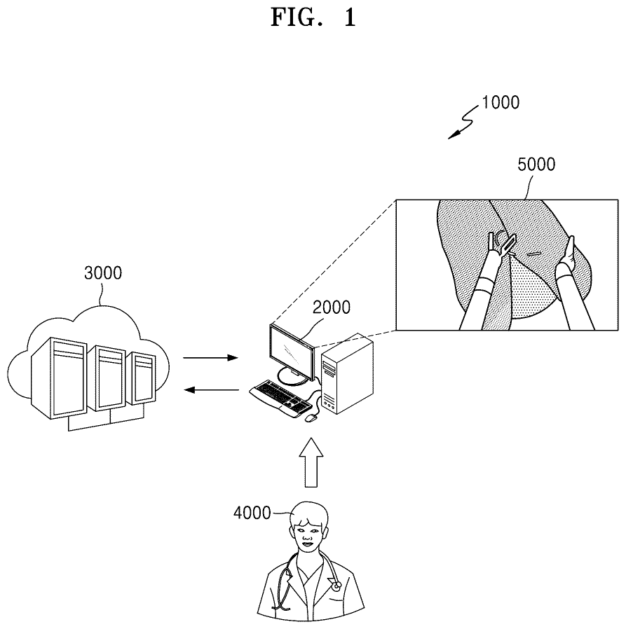

FIG. 1 is a diagram for describing an example of a system for driving a surgical instrument, according to an embodiment.

Referring to FIG. 1, a system 1000 includes a user terminal 2000 and a server 3000. For example, the user terminal 2000 and the server 3000 may be connected to each other in a wired or wireless communication manner to transmit and receive data (e.g., a position of a first robot, a position of a second robot, first position information, second position information, third position information, or driving information) to and from each other.

For convenience of description, FIG. 1 illustrates that the system 1000 includes the user terminal 2000 and the server 3000, but the present disclosure is not limited thereto. For example, other external devices (not shown) may be included in the system 1000, and operations of the user terminal 2000 and the server 3000 to be described below may be implemented by a single device (e.g., the user terminal 2000 or the server 3000) or a plurality of devices.

The user terminal 2000 may be a computing device that is provided with a display device and a device (e.g., a keyboard or a mouse) for receiving an input from a user 4000, and includes a memory and a processor. For example, the display device may be implemented as a touch screen to receive a user input. For example, the user terminal 2000 may correspond to a notebook personal computer (PC), a desktop PC, a laptop, a tablet computer, a smart phone, or the like, but is not limited thereto.

The server 3000 may be a device that communicates with the user terminal 2000 and an external device (not shown). For example, the server 3000 may be a device that stores various pieces of data including manipulation information about a motion of the user 4000, a position of the first robot, a position of the second robot, first position information, second position information, third position information, and the like.

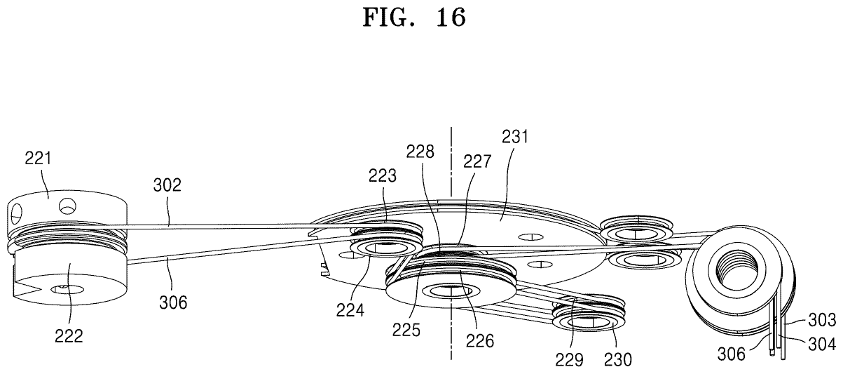

Alternatively, the server 3000 may be a computing device including a memory and a processor, and having computational capabilities. For example, the server 3000 may perform at least some of operations of the user terminal 2000 to be described below with reference to FIGS. 1 to 25. For example, the server 3000 may be a cloud server, but is not limited thereto.

The user terminal 2000 may generate third position information about a relationship between a position of the first robot and a position of the second robot. For example, the user terminal 2000 may generate first position information about a relationship between a position of the first robot and a position of the second robot, based on the position of the first robot, generate second position information about a relationship between the position of the second robot and the position of the first robot, based on the position of the second robot, and generate third position information about a relationship between the position of the first robot and the position of the second robot, based on the first position information and the second position information.

The first position information may include position information about the relationship between the position of the first robot and the position of the second robot, which is generated, based on the position of the first robot. For example, the first position information may include relative position information about the position of the second robot with respect to the position of the first robot. As another example, the first position information may include an angle value between a line connecting the position of the first robot to the position of the second robot, and a first axis that is set based on the position of the first robot.

The first axis may include an axis that is set with respect to a certain direction of the first robot. For example, the first axis may include an axis that is set to be perpendicular to a plane facing a forward direction of the first robot. Here, the forward direction may refer to, but is not limited to, the direction in which robotic arms of the first robot perform surgical motions.

The second position information may include position information about the relationship between the position of the second robot and the position of the first robot, which is generated based on the position of the second robot. For example, the second position information may include relative position information about the position of the first robot with respect to the position of the second robot. As another example, the second position information may include an angle value between a line connecting the position of the second robot to the position of the first robot, and a second axis that is set based on the position of the second robot.

The second axis may include an axis that is set with respect to a certain direction of the second robot. For example, the second axis may include an axis that is set to be perpendicular to a plane facing a forward direction of the second robot. Here, the forward direction may refer to, but is not limited to, the direction in which robotic arms of the second robot perform surgical motions.

The third position information may refer to position information generated based on each of the position of the first robot and the position of the second robot. For example, the third position information may include a relative angle generated based on the first axis including the position of the first robot and the second axis including the position of the second robot.

In addition, the user terminal 2000 may control at least one of the first robot and the second robot based on the third position information.

For example, the user terminal 2000 may generate driving information based on a motion of a master robot controlling the first robot and the second robot, and transformation relationships between the position of the first robot equipped with a camera, the position of the second robot equipped with a surgical instrument, and the position of the surgical instrument. In addition, the user terminal 2000 may control at least one of the first robot and the second robot based on the driving information.

In detail, the user terminal 2000 may generate first intermediate driving information based on a transformation relationship between a position in an image captured by a camera and the position of the first robot, and motion information about the master robot. In addition, the user terminal 2000 may generate second intermediate driving information based on a transformation relationship between the position of the first robot and the position of the second robot, and the first intermediate driving information. In addition, the user terminal 2000 may generate driving information based on a transformation relationship between the position of the surgical instrument and the position of the second robot, and the second intermediate driving information.

For example, when generating the second driving information, the user terminal 2000 may use the transformation relationship between the position of the first robot and the position of the second robot. In detail, the user terminal 2000 may generate second driving information by using information about a transformation matrix from the coordinate system of the second robot equipped with the surgical instrument, to the coordinate system of the first robot equipped with the camera.

Meanwhile, through an application installed in the user terminal 2000, the user terminal 2000 may generate third position information about the relationship between the position of the first robot and the position of the second robot, or drive at least one of the first robot and the second robot. Here, the application may be a software program installed for the user 4000 to drive a surgical instrument (e.g., the surgical instrument mounted on the second robot). For example, through the application, the user 4000 may perform various medical activities, such as generating first position information, generating second position information, generating third position information, or controlling a surgical instrument based on the third position information.

Meanwhile, the user terminal 2000 may output an image 5000 showing a motion of the surgical instrument driven based on a motion of the user 4000. For example, the user terminal 2000 may control a motion of the surgical instrument based on the third position information, and the controlled motion of the surgical instrument may be output through the image 5000 to be viewed by the user 4000. The user 4000 may intuitively understand the motion of the surgical instrument according to the motion of the user 4000 through the image 5000 showing the motion of the surgical instrument, and may manipulate the surgical instrument more accurately.

Meanwhile, for convenience of description, throughout the present specification, it is described that the user terminal 2000 generates first position information about a relationship between the position of the first robot and the position of the second robot, based on the position of the first robot, generates second position information about a relationship between the position of the second robot and the position of the first robot, based on the position of the second robot, and generates third position information about a relationship between the position of the first robot and the position of the second robot, based on the first position information and the second position information, but the present disclosure is not limited thereto. For example, at least some of the operations performed by the user terminal 2000 may be performed by the server 3000.

In other words, at least some of the operations of the user terminal 2000 to be described below with reference to FIGS. 1 to 25 may be performed by the server 3000. For example, the server 3000 may generate first position information about a relationship between the position of the first robot and the position of the second robot, based on the position of the first robot. In addition, the server 3000 may generate second position information about a relationship between the position of the second robot and the position of the first robot, based on the position of the second robot. In addition, the server 3000 may generate third position information about a relationship between the position of the first robot and the position of the second robot, based on the first position information and the second position information.

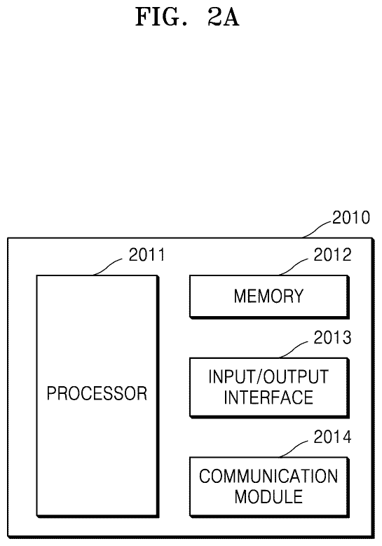

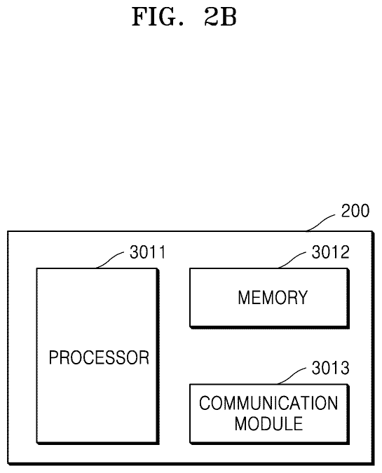

FIG. 2A is a configuration diagram illustrating an example of a user terminal according to an embodiment.

Referring to FIG. 2A, a user terminal 2010 includes a processor 2011, a memory 2012, an input/output interface 2013, and a communication module 2014. For convenience of description, FIG. 2A illustrates only components related to the present disclosure. Thus, other general-purpose components other than the components illustrated in FIG. 2A may be further included in the user terminal 2010. In addition, it is obvious to those of skill in the art related to the present disclosure that the processor 2011, the memory 2012, the input/output interface 2013, and the communication module 2014 illustrated in FIG. 2A may also be implemented as independent devices.

The processor 2011 may process commands of a computer program by performing basic arithmetic, logic, and input/output operations. Here, the commands may be provided from the memory 2012 or an external device (e.g., the server 3000). In addition, the processor 2011 may control the overall operation of other components included in the user terminal 2010.

The processor 2011 may generate first position information about a relationship between the position of a first robot and the position of a second robot, based on the position of the first robot. For example, the processor 2011 may generate first position information including relative position information about the position of the second robot with respect to the position of the first robot. As another example, the processor 2011 may generate first position information including an angle value between a line connecting the position of the first robot to the position of the second robot, and a first axis that is set based on the position of the first robot.

In addition, the processor 2011 may generate second position information about a relationship between the position of the second robot and the position of the first robot, based on the position of the second robot. For example, the processor 2011 may generate second position information including relative position information about the position of the first robot with respect to the position of the second robot. As another example, the processor 2011 may generate second position information including an angle value between a line connecting the position of the second robot to the position of the first robot, and a second axis that is set based on the position of the second robot.

In addition, the processor 2011 may generate third position information about a relationship between the position of the first robot and the position of the second robot, based on the first position information and the second position information. For example, the processor 2011 may generate third position information including a relative angle generated based on the position of the first robot and the position of the second robot.

Meanwhile, the processor 2011 may control at least one of the first robot and the second robot based on the third position information. For example, the processor 2011 may generate driving information based on a motion of a master robot controlling the first robot and the second robot, and transformation relationships between the position of the first robot equipped with a camera, the position of the second robot equipped with a surgical instrument, and the position of the surgical instrument. In addition, the processor 2011 may control at least one of the first robot and the second robot based on the driving information.

For example, the processor 2011 may generate first intermediate driving information based on a transformation relationship between a position in an image captured by a camera and the position of the first robot, and motion information about the master robot. In addition, the processor 2011 may generate second intermediate driving information based on a transformation relationship between the position of the first robot and the position of the second robot, and the first intermediate driving information. In addition, the processor 2011 may generate driving information based on a transformation relationship between the position of the surgical instrument and the position of the second robot, and the second intermediate driving information. In addition, the processor 2011 may control at least one of the first robot and the second robot based on the driving information.

In addition, the processor 2011 may generate motion information about the master robot based on a means for controlling the position and function of the surgical instrument based on a motion of the user. The motion information about the master robot may include manipulation information about a motion of the user. For example, the processor 2011 may generate manipulation information about a motion of the user based on the means for controlling the position and function of the surgical instrument based on a motion of the user.

The means for controlling the position and function of the surgical instrument based on a motion of the user may be in the form of a handle-shaped manipulation member, but is not limited thereto and may be implemented in various shapes to achieve the same purpose. For example, a portion of the means may be formed in the shape of a handle, another portion may be formed in the shape of a clutch button, and a finger insertion tube or the like may be further formed to allow an operator's finger to be inserted and fixed to facilitate manipulation of a surgical tool.

In addition, the means for controlling the position and function of the surgical instrument based on a motion of the user may be a component included in the master robot. For example, the processor 2011 may generate motion information about the master robot based on the means for controlling the position and function of the surgical instrument based on a motion of the user.

The manipulation information refers to information indicating an intuitive motion of the user to control the position and function of the surgical instrument. In detail, the manipulation information may include information about a position and an orientation on a physical coordinate system of a means for allowing a user to control the position and function of a surgical instrument.

Meanwhile, the processor 2011 may generate manipulation information based on position information and orientation information about the means for allowing a user to control the position and function of a surgical instrument. For example, the processor 2011 may generate manipulation information by using a difference between initial position information and initial orientation information about the means for allowing a user to control the position and function of a surgical instrument, and position information and orientation information about the means after a manipulation by the user.

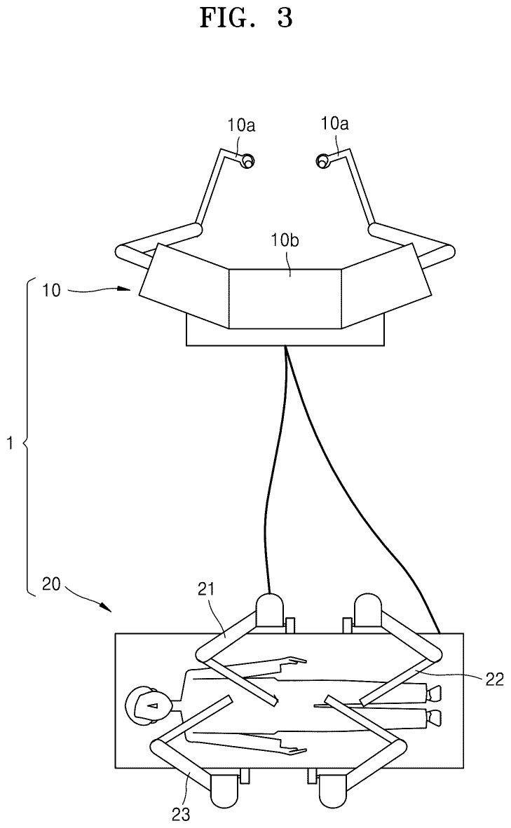

Detailed examples in which the processor 2011 according to an embodiment operates will be described with reference to FIGS. 3 to 25.

The processor 2011 may be implemented as an array of a plurality of logic gates, or may be implemented as a combination of a general-purpose microprocessor and a memory storing a program executable by the microprocessor. For example, the processor 2011 may include a general-purpose processor, a central processing unit (CPU), a microprocessor, a digital signal processor (DSP), a controller, a microcontroller, a state machine, and the like. In some environments, the processor 2011 may include an application-specific integrated circuit (ASIC), a programmable logic device (PLD), a field-programmable gate array (FPGA), and the like. For example, processor 2011 may refer to a combination of processing devices, such as a combination of a DSP and a microprocessor, a combination of a plurality of microprocessors, a combination of one or more microprocessors combined with a DSP core, or a combination of any other such configurations.

The memory 2012 may include any non-transitory computer-readable recording medium. For example, the memory 2012 may include a permanent mass storage device, such as random-access memory (RAM), read-only memory (ROM), a disk drive, a solid-state drive (SSD), or flash memory. As another example, the permanent mass storage device, such as ROM, an SSD, flash memory, or a disk drive, may be a permanent storage device separate from the memory. In addition, the memory 2012 may store an operating system (OS) and at least one piece of program code (e.g., code for the processor 2011 to perform operations to be described below with reference to FIGS. 3 to 25).

These software components may be loaded from a computer-readable recording medium separate from the memory 2012. The separate computer-readable recording medium may be a recording medium that may be directly connected to the user terminal 2010, and may include, for example, a computer-readable recording medium, such as a floppy drive, a disk, a tape, a digital video disc (DVD)/compact disc ROM (CD-ROM) drive, or a memory card. Alternatively, the software components may be loaded into the memory 2012 through the communication module 2014 rather than a computer-readable recording medium. For example, at least one program may be loaded into the memory 2012 on the basis of a computer program (e.g., a computer program for the processor 2011 to perform operations to be described below with reference to FIGS. 3 to 25) installed by files provided via the communication module 2014 by developers or a file distribution system that distributes installation files of applications.

The input/output interface 2013 may be a unit for an interface with a device (e.g., a keyboard or a mouse) for input or output that may be connected to the user terminal 2010 or included in the user terminal 2010. The input/output interface 2013 may be implemented separately from the processor 2011, but is not limited thereto, and may be implemented to be included in the processor 2011.

The communication module 2014 may provide a configuration or function for the server 3000 and the user terminal 2010 to communicate with each other through a network. In addition, the communication module 2014 may provide a configuration or function for the user terminal 2010 to communicate with another external device. For example, a control signal, a command, data, and the like provided under control of the processor 2011 may be transmitted to the server 3000 and/or an external device through the communication module 2014 and a network.

Meanwhile, although not illustrated in FIG. 2A, the user terminal 2010 may further include a display device. For example, the display device may be implemented as a touch screen. Alternatively, the user terminal 2010 may be connected to an independent display device in a wired or wireless communication manner to transmit and receive data to and from the display device. For example, a video or an image for driving a surgical instrument may be provided through the display device by using driving information.

FIG. 2B is a configuration diagram illustrating an example of a server according to an embodiment.

Referring to FIG. 2B, a server 3010 includes a processor 3011, a memory 3012, and a communication module 3013. For convenience of description, FIG. 2B illustrates only components related to the present disclosure. Thus, other general-purpose components other than the components illustrated in FIG. 2B may be further included in the server 3010. In addition, it is obvious to those of skill in the art related to the present disclosure that the processor 3011, the memory 3012, and the communication module 3013 illustrated in FIG. 2B may also be implemented as independent devices.

The processor 3011 may generate first position information about a relationship between the position of a first robot and the position of a second robot, based on the position of the first robot. In addition, the processor 3011 may generate second position information about a relationship between the position of the second robot and the position of the first robot, based on the position of the second robot. In addition, the processor 3011 may generate third position information about a relationship between the position of the first robot and the position of the second robot, based on the first position information and the second position information.

In other words, at least one of the operations of the processor 2011 described above with reference to FIG. 2A may be performed by the processor 3011. In this case, the user terminal 2010 may output, through the display device, information transmitted from the server 3010.

Meanwhile, an implementation example of the processor 3011 is the same as that of the processor 2011 described above with reference to FIG. 2A, and thus, detailed descriptions thereof will be omitted.

Various pieces of data, such as data required for an operation of the processor 3011 or data generated according to an operation of the processor 3011, may be stored in the memory 3012. In addition, the memory 3012 may store an OS and at least one program (e.g., a program required for the processor 3011 to operate).

Meanwhile, an implementation example of the memory 3012 is the same as that of the memory 2012 described above with reference to FIG. 2A, and thus, detailed descriptions thereof will be omitted.

The communication module 3013 may provide a configuration or function for the server 3010 and the user terminal 2010 to communicate with each other through a network. In addition, the communication module 3013 may provide a configuration or function for the server 3010 to communicate with another external device. For example, a control signal, a command, data, and the like provided under control of the processor 3011 may be transmitted to the user terminal 2010 and/or an external device through the communication module 3013 and a network.

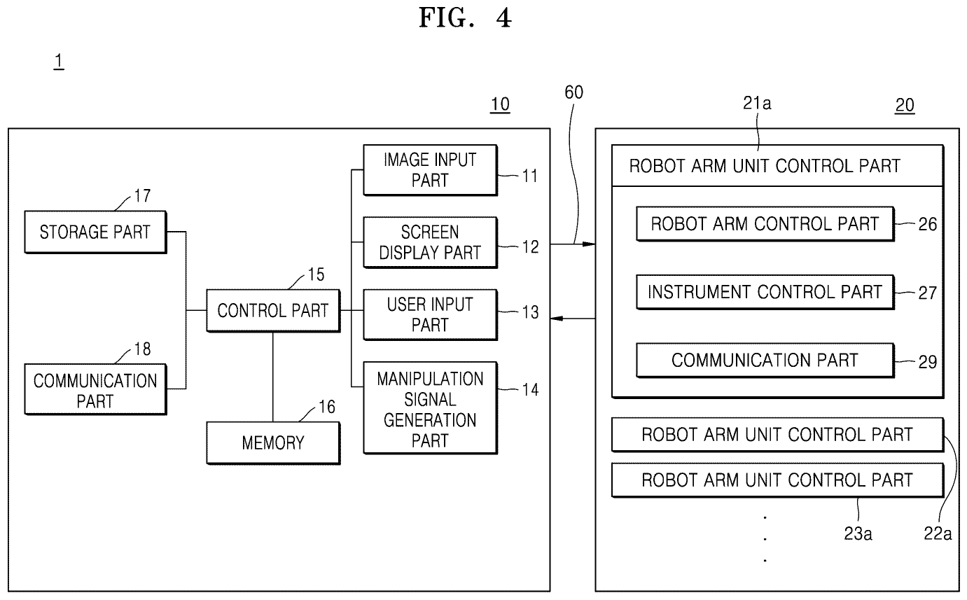

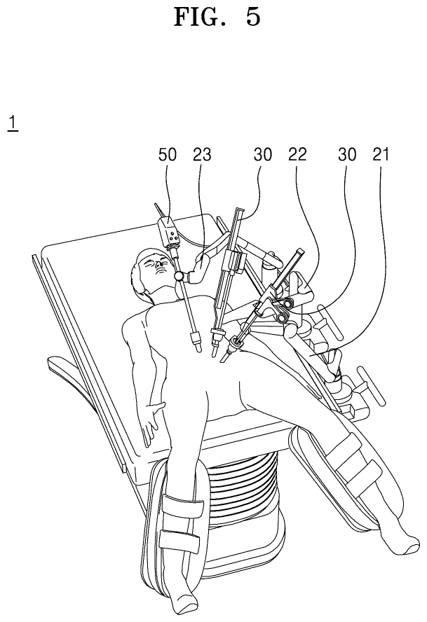

FIG. 3 is a diagram for describing another example of a system for driving a surgical instrument, according to an embodiment, FIG. 4 is a block diagram illustrating an internal configuration of a surgical robotic system of FIG. 3, and FIG. 5 is a perspective view illustrating a slave robot of the surgical robotic system of FIG. 3 and multi-joint surgical instruments mounted on the slave robot.

Referring to FIGS. 3 to 5, a surgical robotic system 1 includes a master robot 10, a slave robot 20, and a multi-joint surgical instrument 30.

The master robot 10 includes a manipulation member 10a and a display member 10b, and the slave robot 20 includes one or more robotic arm units 21, 22, and 23.

The master robot 10 includes the manipulation member 10a to allow an operator to hold and manipulate the manipulation member 10a with both hands. The manipulation member 10a may be implemented with two or more handles as illustrated in FIG. 3, and a manipulation signal according to a manipulation of the handles by the operator is transmitted to the slave robot 20 through a wired or wireless communication network to control the robotic arm units 21, 22, and 23. That is, surgical motions such as positional movement, rotation, and cutting operations of the robotic arm units 21, 22, and 23 may be performed by a manipulation of the handles by the operator. Here, the manipulation signal may include at least one of manipulation information about a motion of the user as described above, and first driving information or second driving information about a motion of a surgical instrument.

For example, the operator may manipulate the robotic arm units 21, 22, and 23 by using a handle-type manipulation lever. The manipulation lever may have various mechanical components depending on its manipulation method, and may be provided in various configurations for operating the robotic arm units 21, 22, and 23 of the slave robot 20 and/or other surgical instruments, such as a master handle for manipulating the motion of the robotic arm units 21, 22, and 23 and various input tools added to the master robot 10 for manipulating the functions of the entire system such as a joystick, a keypad, a trackball, a foot pedal, or a touch screen. Here, the manipulation member 10a is not limited to the shape of a handle and may be applied without any limitation as long as it may control motions of the robotic arm units 21, 22, and 23 through a network such as a wired or wireless communication network.

Meanwhile, according to an embodiment of the present disclosure, manipulation information may be generated based on the manipulation lever or the manipulation member 10a. For example, according to an embodiment of the present disclosure, manipulation information may be generated based on a motion of the user manipulating the manipulation lever or the manipulation member 10a. However, examples of generating manipulation information are not limited to thereto.

Alternatively, a voice input, a motion input, or the like may be applied to the surgical robotic system 1 for user input. That is, the user may wear, on his/her head, glasses or a head-mounted display (HMD) having a sensor attached thereto, and a laparoscope 50 may move according to the direction of the user's gaze. Alternatively, when the user issues a command with a voice, such as “left”, “right”, “first arm”, or “second arm”, the command may be recognized and the corresponding motion may be performed. For example, according to an embodiment of the present disclosure, it is possible to generate manipulation information based on a user's voice, generate first driving information based on the manipulation information, determine, based on the first driving information, whether there is a risk associated with a motion of a surgical instrument, and drive the surgical instrument based on a result of the determination of the risk.

An image captured through the laparoscope 50 is displayed as a screen image on the display member 10b of the master robot 10. In addition, a predetermined virtual manipulation panel may be displayed on the display member 10b independently of or together with the image captured through the laparoscope 50.

The display member 10b may include one or more monitors, and information necessary for surgery may be individually displayed on each monitor. The number of monitors may be determined in various ways depending on the class or type of information required to be displayed.

Meanwhile, the slave robot 20 may include one or more robotic arm units 21, 22, and 23. Here, each of the robotic arm units 21, 22, and 23 may be provided in the form of a module that may operate independently of each other, and in this case, an algorithm for preventing collisions between the robotic arm units 21, 22, and 23 may be applied to the surgical robotic system 1.

In general, a robotic arm has functions similar to a human arm and/or wrist, and refers to a device to which a certain tool may be attached to a wrist area thereof. In the present disclosure, the robotic arm unit 21, 22, or 23 may be defined as a concept that encompasses all components such as an upper arm, a lower arm, a wrist, or an elbow, and a multi-joint surgical instrument or the like coupled to the wrist area. Alternatively, the robotic arm unit 21, 22, or 23 may be defined as a concept that encompasses only components for driving a multi-joint surgical instrument that is coupled to the wrist area, excluding the multi-joint surgical instrument.

As such, the robotic arm units 21, 22, and 23 of the slave robot 20 may be implemented to be driven with multiple degrees of freedom. The robotic arm units 21, 22, and 23 may include, for example a surgical instrument to be inserted into a surgical site of a patient, a yaw driving part for rotating the surgical instrument in a yaw direction according to a surgical position, a pitch driving part for rotating the surgical instrument in a pitch direction orthogonal to the rotation drive of the yaw driving part, a transfer driving part for moving the surgical instrument in the longitudinal direction, a rotation driving part for rotating the surgical instrument, and a surgical instrument driving part for driving an end effector at an end of the surgical instrument to incise or cut a surgical lesion. However, the configurations of the robotic arm units 21, 22, and 23 are not limited thereto, and it should be understood that this example does not limit the scope of the present disclosure. Here, a detailed description of an actual control process, such as rotation or movement of the robotic arm unit 21, 22, or 23 in a corresponding direction when the operator manipulates the manipulation member 10a, will be omitted.

Here, multi-joint surgical instruments 30 may be attached to two of the robotic arm units 21, 22, and 23, and the laparoscope 50 may be attached to the other one of them. In addition, a surgeon may select the robotic arm unit 21, 22, or 23 to be controlled through the master robot 10. As such, the surgeon may directly control a total of three or more surgical instruments through the master robot 10 without the need for a surgical assistant, and thus manipulate various instruments accurately and freely as intended by the surgeon.

Meanwhile, one or more slave robots 20 may be provided to perform a surgery on a patient, and the laparoscope 50 for displaying a surgical site as a screen image through the display member 10b may be implemented as an independent slave robot 20. In addition, as described above, embodiments of the present disclosure may be used universally for surgeries using various surgical endoscopes other than laparoscopes (e.g., thoracoscopes, arthroscopes, or rhinoscopes).

Meanwhile, the master robot 10 may generate manipulation information about a motion of the user for driving a surgical instrument, generate first driving information based on the manipulation information, determine, based on the first driving information, whether there is a risk associated with a motion of the surgical instrument, and drive the surgical instrument based on a result of the determination of the risk. In addition, the master robot 10 may update the first driving information based on the result of the determination of the risk.

For example, the master robot 10 may control the robotic arm units 21, 22, and 23 by transmitting the first driving information to the slave robot 20 through a wired or wireless communication network. That is, surgical motions such as positional movement, rotation, and cutting operations of the robotic arm units 21, 22, and 23 may be performed by a manipulation of the handles by the operator.

Referring to FIG. 4, in an embodiment of the present disclosure, the master robot 10 may include an image input part 11, a screen display part 12, a user input part 13, a manipulation signal generation part 14, a control part 15, a memory 16, a storage part 17, and a communication part 18.

Meanwhile, the master robot 10 may be included in the user terminal of FIG. 2A. For example, the manipulation signal generation part 14, the control part 15, and the like may be included in the processor 2011, the memory 16, the storage part 17, and the like may be included in the memory 2012, and the communication part 18 may be included in the communication module 2014, but examples of the master robot 10 are not limited to thereto.

The image input part 11 may receive, through a wired or wireless communication network, an image captured through a camera provided in the laparoscope 50 of the slave robot 20. The image captured through the camera may include an image representing a motion of a surgical instrument driven by using first driving information or second driving information.

The screen display part 12 outputs, as visual information, a screen image corresponding to the image received through the image input part 11. In addition, when biometric information of a patient is input, the screen display part 12 may further output information corresponding to the biometric information. In addition, the screen display part 12 may further output image data related to the patient's surgical site (e.g., an X-ray image, a computed tomography (CT) image, or a magnetic resonance imaging (MRI) image). Here, the screen display part 12 may be implemented in the form of a display member (see 10b of FIG. 3), and an image process for outputting the received image as a screen image through the screen display part 12 may be performed by the control part 15. Here, the image may include an image representing a motion of a surgical instrument driven by using first driving information or second driving information.

In the embodiment illustrated in FIG. 4, the image input part and the screen display part are illustrated as components included in the master robot 10, but are not limited thereto. The display member may be provided as a separate member spaced apart from the master robot 10. Alternatively, the display member may be provided as a component of the master robot 10. In addition, in another embodiment, a plurality of display members may be provided, one of which may be arranged adjacent to the master robot 10, and some of which may be arranged at a position that is spaced a little apart from the master robot 10.

Here, the screen display part 12 (i.e., the display member 10b of FIG. 3) may be provided as a stereoscopic display device. In detail, the stereoscopic display device refers to an image display device for applying stereoscopic technology to add depth information to a two-dimensional image, and enabling a viewer to feel the liveliness and reality of three dimensions by using the depth information. The surgical robotic system 1 according to an embodiment of the present disclosure may include a stereoscopic display device as the screen display part 12 to provide a more realistic virtual environment to a user.

The user input part 13 is a unit for allowing the operator to control the positions and functions of the robotic arm units 21, 22, and 23 of the slave robot 20. The user input part 13 may be formed in the form of a handle-shaped manipulation member (see 10a of FIG. 3) as illustrated in FIG. 3, but its shape is not limited thereto, and the user input part 13 may be implemented in various shapes to achieve the same purpose. In addition, for example, a portion of the user input unit 13 may be formed in the shape of a handle, and another portion may be formed in the shape of a clutch button, and a finger insertion tube or insertion tube ring may be further formed so as to allow the operator's finger to be inserted and fixed to facilitate manipulation of the surgical tool.

Meanwhile, according to an embodiment of the present disclosure, manipulation information may be generated based on a motion of the operator with respect to the user input part 13. For example, according to an embodiment of the present disclosure, manipulation information may be generated based on a motion of the operator manipulating the user input part 13. However, examples of generating manipulation information are not limited to thereto.

When the operator manipulates the user input part 13 to move the position of the robotic arm unit 21, 22, or 23 or to manipulate a surgical motion thereof, the manipulation signal generation part 14 may generate a manipulation signal corresponding to the manipulation. For example, when the operator manipulates the user input part 13 to move the position of the robotic arm unit 21, 22, or 23 or to manipulate a surgical motion thereof, the manipulation signal generation part 14 may generate manipulation information corresponding to the manipulation.

For example, the manipulation signal generation part 14 transmits the generated manipulation signal to the control part 15, or to the slave robot 20 through the communication part 18. The manipulation signal may be transmitted and received through a wired or wireless communication network. Based on the transmitted manipulation signal, the control part 15 may control the slave robot 20 or the multi-joint surgical instrument 30 to operate. Alternatively, based on the transmitted manipulation signal, a robotic arm control part 26 included in the slave robot 20 may control the robotic arm units 21, 22, and 23 to operate. Alternatively, based on the transmitted manipulation signal, an instrument control part 27 included in the slave robot 20 may control the multi-joint surgical instrument 30 to operate. However, the method by which a motion of the slave robot 20 or the multi-joint surgical instrument 30 is controlled based on a manipulation signal is not limited thereto.

The instrument control part 27 may serve to receive a manipulation signal generated by the manipulation signal generation part 14 of the master robot 10 and control the multi-joint surgical instrument 30 to operate according to the manipulation signal.

The control part 15 is a kind of central processing unit and controls the operation of each component such that the above-described functions may be performed. For example, the control part 15 may perform a function of converting an image input through the image input part 11 into a screen image to be displayed through the screen display part 12. As another example, the control part 15 may generate first driving information about positional movements or surgical motions of the robotic arm units 21, 22, and 23, based on the manipulation information. In addition, the control part 15 may determine whether there is a risk associated with the positional movements or surgical motions of the robotic arm units 21, 22, and 23, based on the first driving information. In addition, the control part 15 may update the first driving information based on a result of the determination of the risk. In addition, the control part 15 may drive the robotic arm units 21, 22, and 23 based on the result of the determination of the risk. In addition, the control part 15 may generate second driving information based on the first driving information, and drive the robotic arm units 21, 22, and 23 based on the second driving information.

Meanwhile, it is described above that the control part 15 generates the first driving information, updates the first driving information, or generates the second driving information based on the first driving information, but the present disclosure is not limited thereto, and the operations may be performed by other control parts (e.g., the robotic arm control part 26, or the instrument control part 27) according to the present disclosure.

The memory 16 may perform a function of temporarily or permanently storing data processed by the control part 15. Here, the memory 16 may include a magnetic storage medium or a flash storage medium, but the scope of the present disclosure is not limited thereto.

The storage part 17 may store data received from the slave robot 20. In addition, the storage part 17 may store various types of input data (e.g., patient data, device data, or surgery data).

The communication part 18 provides a communication interface necessary to transmit and receive image data transmitted from the slave robot 20 and control data transmitted from the master robot 10, in conjunction with a communication network 60. The image data transmitted from the slave robot 20 may include an image representing a motion of a surgical instrument driven by using first driving information or second driving information. The control data transmitted from the master robot 10 may include first driving information or second driving information about a motion of the slave robot 20.

The slave robot 20 includes a plurality of robotic arm unit control parts 21a, 22a, and 23a. In addition, the robotic arm unit control part 21a includes the robotic arm control part 26, the instrument control part 27, and a communication part 29. In addition, the robotic arm unit control part 21a may further include a rail control part 28.

The robotic arm control part 26 may serve to receive a manipulation signal generated by the manipulation signal generation part 14 of the master robot 10 and control the robotic arm units 21, 22, and 23 to operate according to the manipulation signal. For example, the robotic arm control part 26 may serve to receive the generated (or updated) first driving information or second driving information from the master robot 10, and control the robotic arm units 21, 22, and 23 to operate according to the first driving information or the second driving information.

The instrument control part 27 may serve to receive a manipulation signal generated by the manipulation signal generation part 14 of the master robot 10 and control the multi-joint surgical instrument 30 to operate according to the manipulation signal. For example, the instrument control part 27 may serve to receive the generated (or updated) first driving information from the master robot 10, and control the multi-joint surgical instrument 30 to operate according to the first driving information.

The communication part 29 provides a communication interface necessary to transmit and receive image data transmitted from the slave robot 20 and control data transmitted from the master robot 10, in conjunction with the communication network 60. The image data transmitted from the slave robot 20 may include an image representing a motion of a surgical instrument driven by using first driving information or second driving information. The control data transmitted from the master robot 10 may include first driving information or second driving information about a motion of the slave robot 20.

Meanwhile, the communication network 60 serves to connect the master robot 10 to the slave robot 20. That is, the communication network 60 refers to a communication network for providing a connection path such that the master robot 10 and the slave robot 20 may transmit and receive data to and from each other after being connected to each other. The communication network 60 may include, for example, a wired network such as a local area network (LAN), a wide area network (WAN), a metropolitan area network (MAN), or an integrated services digital network (ISDN), or a wireless network such as a wireless LAN (WLAN), code-division multiple access (CDMA), Bluetooth, or satellite communication, but the scope of the present disclosure is not limited thereto.

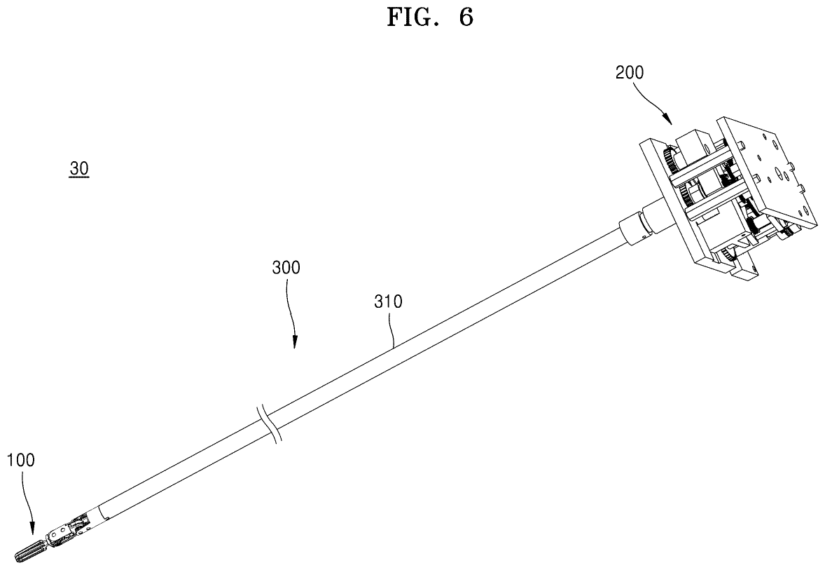

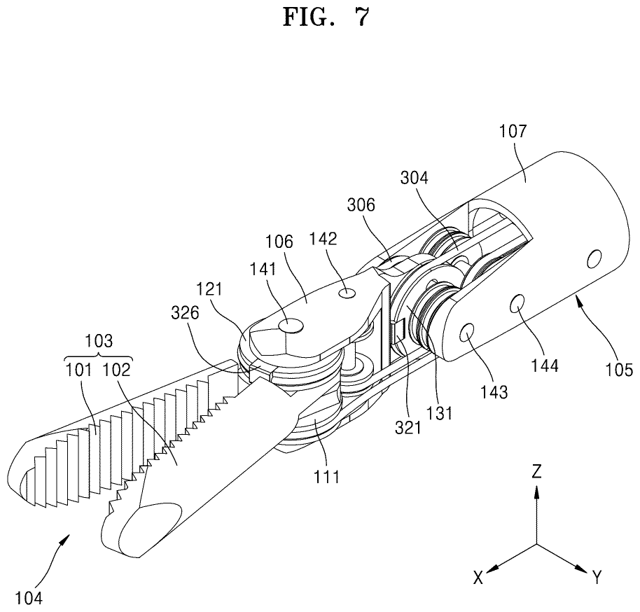

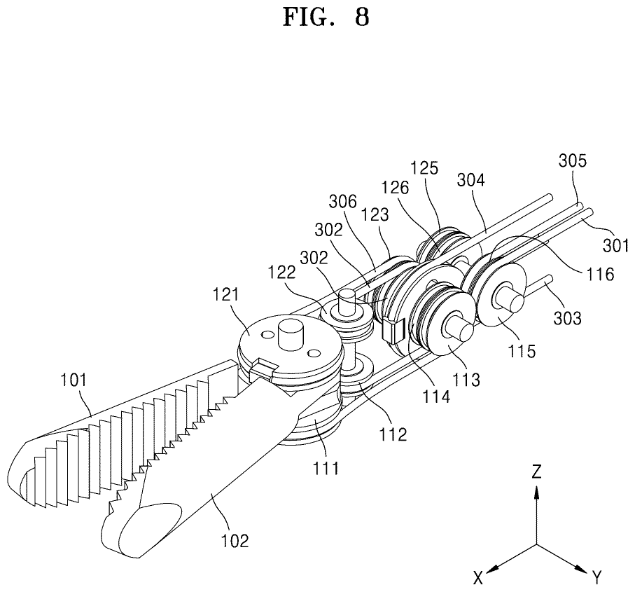

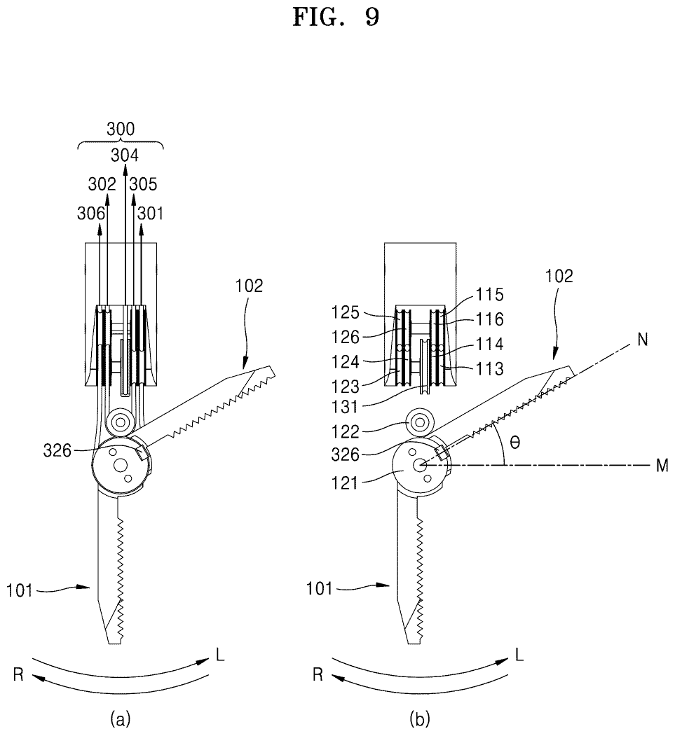

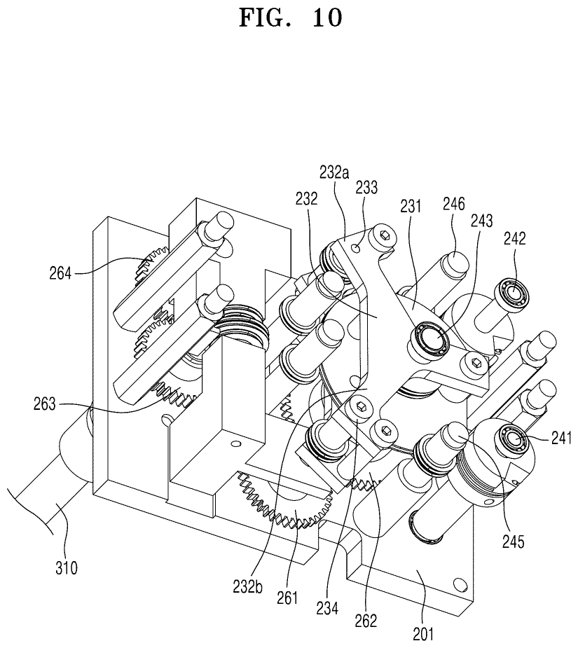

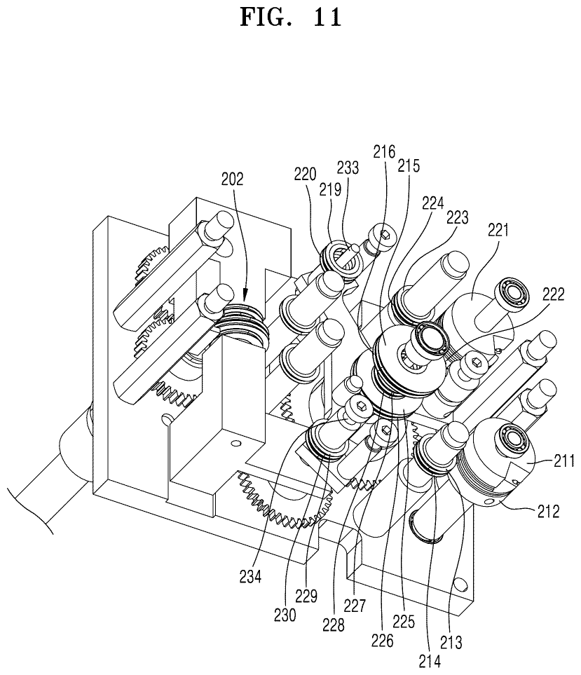

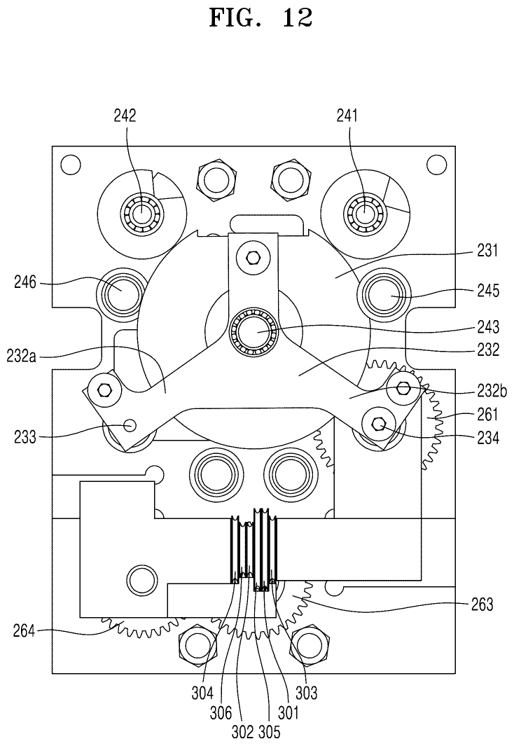

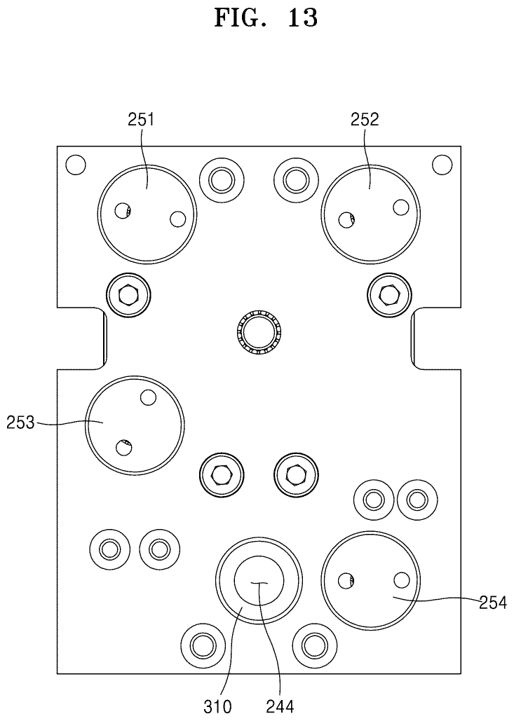

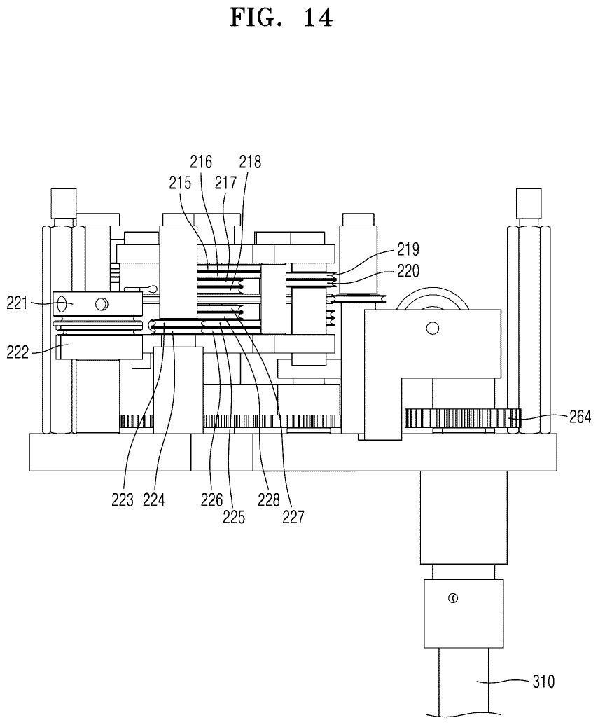

FIG. 6 is a perspective view illustrating a multi-joint surgical instrument according to an embodiment of the present disclosure, FIGS. 7 and 8 are perspective views of an end tool of the multi-joint surgical instrument of FIG. 6, and FIG. 9 is a plan view of the end tool of the multi-joint surgical instrument of FIG. 6. FIGS. 10 and 11 are perspective views of a driving part of the multi-joint surgical instrument of FIG. 6, FIG. 12 is a plan view of the driving part of the multi-joint surgical instrument of FIG. 6, FIG. 13 is a rear view of the driving part of the multi-joint surgical instrument of FIG. 6, and FIG. 14 is a side view of the driving part of the multi-joint surgical instrument of FIG. 6.

First, referring to FIG. 6, the multi-joint surgical instrument 30 according to an embodiment of the present disclosure may include an end tool 100, a driving part 200, and a power transmission part 300, and the power transmission part 300 may include a connection part 310.

The connection part 310 may be formed in the shape of a hollow shaft to accommodate one or more wires (to be described below) therein, and the driving part 200 may be coupled to one end of the connection part 310, and the end tool 100 is coupled to the other end of the connection part 310 such that the connection part 310 serves to connect the driving part 200 to the end tool 100.

The driving part 200 is formed at one end of the connection part 310 and provides an interface that may be coupled to a robotic arm unit (see 21 of FIG. 3 and the like). Thus, when a master robot (see 10 of FIG. 3) is operated by a user, a motor (not shown) of the robotic arm unit (see 21 of FIG. 3 and the like) operates to allow the end tool 100 of the multi-joint surgical instrument 30 to perform a corresponding motion, and a driving force of the motor (not shown) is transmitted to the end tool 100 through the driving part 200. In other words, it may also be described that the driving part 200 itself serves as an interface connecting the multi-joint surgical instrument 30 to the slave robot 20.

For example, when a user input part (see 13 of FIG. 3) is operated by the user, the motor (not shown) of the robotic arm unit (see 21 of FIG. 3 and the like) may operate to allow the end tool 100 of the multi-joint surgical instrument 30 to perform a corresponding motion, and a driving force of the motor (not shown) may be transmitted to the end tool 100 through the driving part 200.

The end tool 100 is formed at the other end of the connection part 310, and is inserted into a surgical site to perform a motion necessary for surgery. As an example of the end tool 100, a pair of jaws 101 and 102 for performing a grip motion may be used as illustrated in FIG. 7. However, an embodiment of the present disclosure is not limited thereto, and various surgical devices may be used as the end tool 100. For example, a one-armed cautery may be used as the end tool. As the end tool 100 is connected to the driving part 200 by the power transmission part 300, the end tool 100 receives a driving force of the driving part 200 transmitted through the power transmission part 300 to perform motions necessary for surgery, such as a grip motion, a cutting motion, or a suturing motion.

Here, the end tool 100 of the multi-joint surgical instrument 30 according to an embodiment of the present disclosure may be formed to be rotatable in two or more directions, and for example, the end tool 100 may be formed to perform a pitch motion around a rotation shaft 143 of FIG. 7 and simultaneously perform a yaw motion and an actuation motion around a rotation shaft 141 of FIG. 7.

Here, each of the pitch motion, the yaw motion, and the actuation motion used in the present disclosure is defined as follows.

First, the pitch motion refers to a motion of the end tool 100 rotating in a vertical direction with respect to an extension direction of the connection part 310 (the X-axis direction of FIG. 7), that is, a motion of rotating around the Y-axis of FIG. 7. In other words, the pitch motion refers to a motion that the end tool 100 extending from the connection part 310 in the direction in which the connection part 310 extends (the X-axis direction of FIG. 7) rotates up and down around the Y-axis with respect to the connection part 310.

Next, the yaw motion refers to a motion of the end tool 100 rotating in a horizontal direction, that is, a motion of rotating around the Z-axis of FIG. 7, with respect to the extension direction of the connection part 310 (the X-axis direction of FIG. 7). In other words, the yaw motion refers to a motion that the end tool 100 extending from the connection part 310 in the direction in which the connection part 310 extends (the X-axis direction of FIG. 7) rotates left and right around the Z-axis with respect to the connection part 310. That is, the yaw motion refers to a motion of the two jaws 101 and 102, which are formed on the end tool 100, rotating around the Z-axis in the same direction.

In addition, the actuation motion refers to a motion of the end tool 100 rotating around the same rotation axis as that of the yaw motion, but with the two jaws 101 and 102 rotating in the opposite directions to be closed or opened. That is, the actuation motion refers to a motion of the two jaws 101 and 102, which are formed on the end tool 100, rotating around the Z-axis in the opposite directions.

In other words, yaw rotation may refer to a motion of an end tool jaw pulley, which will be described below, rotating around the rotation shaft 141, which is an end tool jaw pulley rotation shaft, and pitch rotation may refer to a motion of the end tool jaw pulley revolving around the rotation shaft 143, which is an end tool pitch rotation shaft.

The roll motion refers to a motion of the multi-joint surgical instrument rotating around the connection part 310. For example, the roll motion may be a motion of the multi-joint surgical instrument rotating clockwise or counterclockwise around the extension direction of the connection part 310 (the X-axis direction of FIG. 7).

In addition, the roll motion may refer to a motion of the end tool 100 rotating around the X-axis with respect to the connection part 310. For example, the roll motion may be a motion of the end tool rotating clockwise or counterclockwise around the extension direction of the connection part 310 (the X-axis direction of FIG. 7).

The power transmission part 300 may serve to connect the driving part 200 to the end tool 100 to transmit the driving force of the driving part 200 to the end tool 100, and may include a plurality of wires, pulleys, links, joints, gears, and the like.

Hereinafter, the end tool 100, the driving part 200, the power transmission part 300, and the like of the multi-joint surgical instrument 30 of FIG. 6 will be described in more detail.

Hereinafter, the power transmission part 300 of the multi-joint surgical instrument 30 of FIG. 6 will be described in more detail.

Referring to FIGS. 6 to 14, the power transmission part 300 of the multi-joint surgical instrument 30 according to an embodiment of the present disclosure may include a wire 301, a wire 302, a wire 303, a wire 304, a wire 305, and a wire 306.

Here, the wire 301 and the wire 305 may be paired to serve as first jaw wires. The wire 302 and the wire 306 may be paired to serve as second jaw wires. Here, components including the wire 301 and the wire 305, which are first jaw wires, and the wire 302 and the wire 306, which are second jaw wires, may be referred to as jaw wires. In addition, the wire 303 and the wire 304 may be paired to serve as pitch wires.

Here, the drawings illustrate that a pair of wires are associated with a rotational motion of the first jaw 101, and another pair of wires are associated with a rotational motion of the second jaw 102, but an embodiment of the present disclosure is not limited thereto. For example, a pair of wires may be associated with the yaw motion, and another pair of wires may be associated with the actuation motion.

In addition, the power transmission part 300 of the multi-joint surgical instrument 30 according to an embodiment of the present disclosure may include a fastening member 321, a fastening member 326, and the like, which are coupled to ends of the respective wires to couple the wires to pulleys. Here, each of the fastening members may have various shapes as necessary, such as a ball shape or a tube shape.

Here, the fastening member 321, which is a pitch wire fastening member, may be coupled to ends of the wire 303 and the wire 304, which are the pitch wires, on the side of the end tool 100, to serve as a pitch wire-end tool fastening member. In addition, although not illustrated in the drawings, a pitch wire-driving part fastening member (not shown) may be coupled to ends of the wire 303 and the wire 304, which are pitch wires, on the side of the driving part 200.

In addition, the fastening member 326, which is a second jaw wire fastening member, may be coupled to ends of the wire 302 and the wire 306, which are second jaw wires, on the side of the end tool 100, to serve as a second jaw wire-end tool fastening member. In addition, although not illustrated in the drawings, a second jaw wire-driving part fastening member (not shown) may be coupled to ends of the wire 302 and the wire 306, which are second jaw wires, on the side of the driving part 200.

In addition, although not illustrated in the drawings, a fastening member (not shown) having the same shape as the fastening member 326 may be coupled to ends of the wire 301 and the wire 305, which are first jaw wires, on the side of the end tool 100, to serve as a first jaw wire-end tool fastening member. In addition, although not illustrated in the drawings, a first jaw wire-driving part fastening member (not shown) may be coupled to ends of the wire 301 and the wire 305, which are first jaw wires, on the side of the driving part 200.

Here, the fastening members are classified as being included in the power transmission part 300, but the fastening members on the side of the end tool 100 may be classified as being included in the end tool 100, and the fastening members on the side of the driving part 200 may be classified as being included in the driving part 200.

The coupling relationships between the wires, the fastening members, and each pulley may be described in detail as follows.

First, the wire 302 and the wire 306, which are second jaw wires, may be a single wire. When the fastening member 326, which is a second jaw wire-end tool fastening member, is fit into a middle point of the single second jaw wire, and the fastening member 326 is fixed through crimping, both strands of the second jaw wire around the fastening member 326 may be referred to as the wire 302 and the wire 306, respectively.

Alternatively, the wire 302 and the wire 306, which are second jaw wires, may be formed as separate wires and then connected to each other by the fastening member 326.

Then, by coupling the fastening member 326 to a pulley 121, the wire 302 and the wire 306 may be fixedly coupled to the pulley 121. Accordingly, the pulley 121 may rotate as the wire 302 and the wire 306 are pulled and released.

In addition, a second jaw wire-driving part fastening member (not shown) may be coupled to ends of the wire 302 and the wire 306 that are opposite to the ends to which the fastening member 326 is coupled. That is, by fitting the opposite ends of the wire 302 and the wire 306 into the second jaw wire-driving part fastening member (not shown), and then crimping the fastening member (not shown), each of the wire 302 and the wire 306 may be fixed to the second jaw wire-driving part fastening member (not shown).

In addition, by coupling the second jaw wire-driving part fastening member (not shown), which is coupled to the wire 302 and the wire 306, to each of a pulley 221 and a pulley 222, the wire 302 and the wire 306 may be fixedly coupled to the pulley 221 and the pulley 222, respectively. Accordingly, when the pulley 221 and the pulley 222 is rotated by a motor or a human force, the pulley 121 of the end tool 100 may be rotated as the wire 302 and the wire 306 are pulled and released.

Here, a driving part second jaw pulley may include two pulleys, i.e., the pulley 221 and the pulley 222, and thus, the second jaw wire-driving part fastening member may also include two fastening members. Alternatively, the driving part second jaw pulley may include one pulley, the second jaw wire-driving part fastening member may also include one fastening member, and the wire 302 and the wire 306 may be coupled to one fastening member and thus coupled to one driving part second jaw pulley.

In the same manner, the wire 301 and the wire 305, which are first jaw wires, are coupled to the first jaw wire-end tool fastening member (not shown) and the first jaw wire-driving part fastening member (not shown), respectively. In addition, the first jaw wire-end tool fastening member (not shown) is coupled to a pulley 111, and the first jaw wire-driving part fastening member (not shown) is coupled to a pulley 211 and a pulley 212. Accordingly, when the pulley 211 and the pulley 212 is rotated by a motor or a human force, the pulley 111 of the end tool 100 may be rotated as the wire 301 and the wire 305 are pulled and released.

In the same manner, ends of the wire 303 and the wire 304, which are pitch wires, are coupled to the fastening member 321, which is a pitch wire-end tool fastening member, and the other ends of the wire 303 and the wire 304 are coupled to the pitch wire-driving part fastening member (not shown). In addition, the fastening member 321 is coupled to a pulley 131, and the pitch wire-driving part fastening member (not shown) is coupled to a pulley 231. Accordingly, when the pulley 231 is rotated by a motor or a human force, the pulley 131 of the end tool 100 may be rotated as the wire 303 and the wire 304 are pulled and released.

Accordingly, the wire 301 and the wire 305, which are both strands of the first jaw wire, may be coupled to a fastening member 323, which is a first jaw wire-end tool fastening member, and the first jaw wire-driving part fastening member (not shown), so as to form a closed loop together. Similarly, each of the second jaw wire and the pitch wire may be formed to form a closed loop.

Hereinafter, the end tool 100 of the multi-joint surgical instrument 30 of FIG. 6 will be described in more detail.

FIGS. 7 and 8 are perspective views illustrating the end tool of the multi-joint surgical instrument of FIG. 6, and FIG. 9 is a plan view illustrating the end tool of the multi-joint surgical instrument of FIG. 6. Here, FIG. 7 illustrates a state in which an end tool hub 106 and a pitch hub 107 are coupled to the end tool, and FIG. 8 illustrates a state in which the end tool hub 106 and the pitch hub 107 are removed from the end tool.

Referring to FIGS. 7, 8, and 9, the end tool 100 of an embodiment of the present disclosure includes a pair of jaws for performing a grip motion, that is, the first jaw 101 and the second jaw 102. Here, a component encompassing each of the first jaw 101 and the second jaw 102 or both the first jaw 101 and the second jaw 102 may be referred to as a jaw 103.

In addition, the end tool 100 may include the pulley 111, a pulley 112, a pulley 113, a pulley 114, a pulley 115, and a pulley 116, which are associated with a rotational motion of the first jaw 101. In addition, the end tool 100 may include the pulley 121, a pulley 122, a pulley 123, a pulley 124, a pulley 125, and a pulley 126, which are associated with a rotational motion of the second jaw 102.

Here, the drawings illustrate that a group of pulleys are associated with the rotational motion of the first jaw 101, and another group of pulleys are associated with the rotational motion of the second jaw 102, but an embodiment of the present disclosure is not limited thereto. For example, one group of pulleys within the end tool may be associated with a yaw motion, and another group of pulleys may be associated with an actuation motion. Here, the pulleys included in the end tool 100, including the pulleys described above, may be collectively referred to as end tool pulleys.

In addition, although the drawings illustrate that the pulleys facing each other are arranged in parallel with each other, an embodiment of the present disclosure is not limited thereto, and the pulleys may be formed in various positions and sizes suitable for the configuration of the end tool.

In addition, the end tool 100 of an embodiment of the present disclosure may include the end tool hub 106 and the pitch hub 107.

The rotation shaft 141 and a rotation shaft 142, which will be described below, may be inserted through the end tool hub 106, and the end tool hub 106 may accommodate therein at least portions of the first jaw 101 and the second jaw 102, which are axially coupled to the rotation shaft 141. In addition, the end tool hub 106 may accommodate therein at least portions of the pulley 112 and the pulley 122, which are axially coupled to the rotation shaft 142.

In addition, the pulley 131 serving as an end tool pitch pulley may be formed at one end of the end tool hub 106. As illustrated in FIG. 7, the pulley 131 may be formed as a separate member from the end tool hub 106, and then coupled to the end tool hub 106. Alternatively, although not illustrated in the drawings, the pulley 131 may be formed with the end tool hub 106 as one body. That is, one end of the end tool hub 106 may be formed in a disk shape or a semicircular shape like a pulley, and a groove around which a wire may be wound may be formed on an outer circumferential surface of the end tool hub 106. The wire 303 and the wire 304 described above may be coupled to the pulley 131 serving as an end tool pitch pulley, and a pitch motion may be performed as the pulley 131 rotates around the rotation shaft 143.

The rotation shaft 143 and a rotation shaft 144, which will be described below, may be inserted through the pitch hub 107, and the pitch hub 107 may be axially coupled to the end tool hub 106 and the pulley 131 by the rotation shaft 143. Thus, the end tool hub 106 and the pulley 131 (coupled to the end tool hub 106) may be formed to be rotatable around the rotation shaft 143 with respect to the pitch hub 107.

In addition, the pitch hub 107 may accommodate therein at least portions of the pulley 113, the pulley 114, the pulley 123, and the pulley 124, which are axially coupled to the rotation shaft 143. In addition, the pitch hub 107 may accommodate therein at least portions of the pulley 115, the pulley 116, the pulley 125, and the pulley 126, which are axially coupled to the rotation shaft 144.

Further, the end tool 100 of an embodiment of the present disclosure may include the rotation shaft 141, the rotation shaft 142, the rotation shaft 143, and the rotation shaft 144. As described above, the rotation shaft 141 and the rotation shaft 142 may be inserted through the end tool hub 106, and the rotation shaft 143 and the rotation shaft 144 may be inserted through the pitch hub 107.

The rotation shaft 141, the rotation shaft 142, the rotation shaft 143, and the rotation shaft 144 may be arranged sequentially from a distal end 104 of the end tool 100 toward a proximal end 105. Accordingly, starting from the distal end 104, the rotation shaft 141 may be referred to as a first pin, the rotation shaft 142 may be referred to as a second pin, the rotation shaft 143 may be referred to as a third pin, and the rotation shaft 144 may be referred to as a fourth pin.

Here, the rotation shaft 141 may function as an end tool jaw pulley rotation shaft, the rotation shaft 142 may function as an end tool jaw auxiliary pulley rotation shaft, the rotation shaft 143 may function as an end tool pitch rotation shaft, and the rotation shaft 144 may function as an end tool pitch auxiliary rotation shaft of the end tool 100.

One or more pulleys may be fit into each of the rotation shafts 141, 142, 143, and 144, which will be described in detail below.

The pulley 111 functions as an end tool first jaw pulley, the pulley 121 functions as an end tool second jaw pulley, and these two components may be collectively referred to as end tool jaw pulleys.

The pulley 111 and the pulley 121, which are end tool jaw pulleys, are formed to face each other, and are formed to be rotatable independently of each other around the rotation shaft 141, which is an end tool jaw pulley rotation shaft. Here, the drawings illustrate that the pulley 111 and the pulley 121 are formed to be rotated around one rotation shaft 141, but it is needless to say that each jaw pulley may be formed to be rotatable around a separate shaft. Here, the first jaw 101 may be fixedly coupled to the pulley 111 to rotate together with the pulley 111, and the second jaw 102 may be fixedly coupled to the pulley 121 to rotate together with the pulley 121. A yaw motion and an actuation motion of the end tool 100 are performed according to rotation of the pulley 111 and the pulley 121. That is, when the pulley 111 and the pulley 121 rotate in the same direction around the rotation shaft 141, the yaw motion is performed, and when the pulley 111 and the pulley 121 rotate in opposite directions around the rotation shaft 141, the actuation motion is performed.

Here, the first jaw 101 and the pulley 111 may be formed as separate members and then coupled to each other, or the first jaw 101 and the pulley 111 may be formed as one body. Similarly, the second jaw 102 and the pulley 121 may be formed as separate members and then coupled to each other, or the second jaw 102 and the pulley 121 may be formed as one body.

The pulley 112 functions as an end tool first jaw auxiliary pulley, the pulley 122 functions as an end tool second jaw auxiliary pulley, and these two components may be collectively referred to as end tool jaw auxiliary pulleys.

In detail, the pulley 112 and the pulley 122, which are end tool jaw auxiliary pulleys, may be additionally provided on one side of the pulley 111 and the pulley 121. In other words, the pulley 112, which is an auxiliary pulley, may be arranged between the pulley 111 and the pulley 113/pulley 114. In addition, the pulley 122, which is an auxiliary pulley, may be arranged between the pulley 121 and the pulley 123/pulley 124. The pulley 112 and the pulley 122 may be formed to be rotatable independently of each other around the rotation shaft 142. Here, the drawings illustrate that the pulley 112 and the pulley 122 are formed to be rotated around one rotation shaft 142, but it is needless to say that the pulley 112 and the pulley 122 may be formed to be rotatable around separate shafts, respectively. The auxiliary pulleys will be described in more detail below.

The pulley 113 and the pulley 114 may function as end tool first jaw pitch main pulleys, the pulley 123 and the pulley 124 may function as end tool second jaw pitch main pulleys, and these components may collectively be referred to as end tool jaw pitch main pulleys.

The pulley 115 and the pulley 116 may function as end tool first jaw pitch sub-pulleys, the pulley 125 and the pulley 126 may function as end tool second jaw pitch sub-pulleys, and these components may collectively be referred to as end tool jaw pitch sub-pulleys.

Hereinafter, components associated with rotation of the pulley 111 will be described.

The pulley 113 and the pulley 114 may function as end tool first jaw pitch main pulleys. That is, the pulley 113 and the pulley 114 function as main rotation pulleys for a pitch motion of the first jaw 101. Here, the wire 301, which is a first jaw wire, is wound around the pulley 113, and the wire 305, which is another first jaw wire, is wound around the pulley 114.

The pulley 115 and the pulley 116 function as end tool first jaw sub-pulleys. That is, the pulley 115 and the pulley 116 function as sub-rotation pulleys for a pitch motion of the first jaw 101. Here, the wire 301, which is a first jaw wire, is wound around the pulley 115, and the wire 305, which is another first jaw wire, is wound around the pulley 116.

Here, on one side of the pulley 111 and the pulley 112, the pulley 113 and the pulley 114 are arranged to face each other. Here, the pulley 113 and the pulley 114 are formed to be rotatable independently of each other around the rotation shaft 143, which is an end tool pitch rotation shaft. In addition, the pulley 115 and the pulley 116 are arranged on one sides of the pulley 113 and the pulley 114, respectively, to face each other. Here, the pulley 115 and the pulley 116 are formed to be rotatable independently of each other around the rotation shaft 144, which is an end tool pitch auxiliary rotation shaft. Here, the drawings illustrate that the pulley 113, the pulley 115, the pulley 114, and the pulley 116 are formed to be rotatable around the Y-axis direction, but an embodiment of the present disclosure is not limited thereto, and the rotation shafts of the respective pulleys may be formed in various directions suitable for their configurations.

The wire 301, which is a first jaw wire, is wound sequentially around the pulley 115, the pulley 113, and the pulley 111 such that at least portions of the wire 301 come into contact with the pulleys. In addition, the wire 305 connected to the wire 301 by the fastening member 323 is wound to sequentially come into contact with at least portions of the pulley 111, the pulley 112, the pulley 114, and the pulley 116.

In other words, the wire 301 and the wire 305, which are the first jaw wires, are wound to sequentially come into contact with at least portions of the pulley 115, the pulley 113, the pulley 111, the pulley 112, the pulley 114, and the pulley 116, and are formed to move along the above pulleys while rotating the above pulleys.

Accordingly, when the wire 301 is pulled in the direction of an arrow 301 of FIG. 9, the fastening member (not shown) to which the wire 301 is coupled and the pulley 111 coupled to the fastening member (not shown) rotate in the direction of an arrow L of FIG. 9. On the contrary, when the wire 305 is pulled in the direction of an arrow 305 of FIG. 9, the fastening member (not shown) to which the wire 305 is coupled and the pulley 111 coupled to the fastening member (not shown) rotate in the direction of an arrow R of FIG. 9.

Hereinafter, the pulley 112 and the pulley 122 serving as auxiliary pulleys will be described in more detail.

The pulley 112 and the pulley 122 may come into contact with the wire 305, which are first jaw wires, and the wire 302, which is a second jaw wire, to change the arrangement path of the wire 305 and the wire 302 to a certain extent, and thus perform a function of increasing a rotation angle of each of the first jaw 101 and the second jaw 102.

That is, in a case in which no auxiliary pulley is arranged, each of the first jaw and the second jaw may rotate up to the right angle, however, in an embodiment of the present disclosure, by additionally arranging the pulley 112 and the pulley 122, which are auxiliary pulleys, the maximum rotation angle may be increased by θ as illustrated in FIG. 9. This enables an opening motion of the two jaws of the end tool 100 for an actuation motion in a state in which the two jaws are yaw-rotated by 90° in the L direction. This is because the second jaw 102 may be rotated by the additional angle θ as illustrated in FIG. 9. Similarly, the actuation motion may be performed even in a state in which the two jaws are yaw-rotated in the R direction. In other words, a feature of increasing the range of yaw rotation in which an actuation motion is possible may be obtained through the pulley 112 and the pulley 122.

This will be described in more detail as follows.

In a case in which no auxiliary pulley is arranged, as the first jaw wire is fixedly coupled to the end tool first jaw pulley, and the second jaw wire is fixedly coupled to the end tool second jaw pulley, each of the end tool first jaw pulley and the end tool second jaw pulley may rotate only up to 90°. In this case, when an actuation motion is performed in a state in which the first jaw and the second jaw are located at a 90° line, the first jaw may be opened, but the second jaw may not rotate beyond 90°. Thus, there was a problem that, in a state in which the first jaw and the second jaw perform a yaw motion over a certain angle, the actuation motion is not seamlessly performed.

In order to address such a problem, in the multi-joint surgical instrument 30 according to an embodiment of the present disclosure, the pulley 112 and the pulley 122, which are auxiliary pulleys, are additionally arranged on one sides of the pulley 111 and the pulley 121, respectively. As described above, as the arrangement path of the wire 305, which is a first jaw wire, and the wire 302, which is a second jaw wire, are changed to a certain extent by arranging the pulley 112 and the pulley 122, a tangential direction of the wire 305 and the wire 302 is changed, and accordingly, the fastening member 326 for coupling the wire 302 and the pulley 121 is able to rotate up to a line N of FIG. 9. That is, the fastening member 326, which is a coupling part of the wire 302 and the pulley 121, is rotatable until the fastening member 326 is located on a common internal tangent of the pulley 121 and the pulley 122. Similarly, the fastening member 323, which is a coupling part of the wire 305 and the pulley 111, is rotatable until the fastening member 323 is located on a common internal tangent of the pulley 111 and the pulley 112, such that the range of rotation in the L direction may be increased.

In other words, the wire 301 and the wire 305, which are two strands of the first jaw wire wound around the pulley 111 by the pulley 112, are arranged at one side with respect to a plane perpendicular to the Y-axis and passing through the X-axis. Simultaneously, the wire 302 and the wire 306, which are two strands of the second jaw wire wound around the pulley 121 by the pulley 122, are arranged at the other side with respect to the plane perpendicular to the Y-axis and passing through the X-axis.

In other words, the pulley 113 and the pulley 114 are arranged on one side with respect to the plane perpendicular to the Y-axis and passing through the X-axis, and the pulley 123 and the pulley 124 are arranged on the other side with respect to the plane perpendicular to the Y-axis and passing through the X-axis.

In other words, the wire 305 is located on the internal tangent of the pulley 111 and the pulley 112, and the rotation angle of the pulley 111 is increased by the pulley 112. In addition, the wire 302 is located on the internal tangent of the pulley 121 and the pulley 122, and the rotation angle of the pulley 121 is increased by the pulley 122.

According to an embodiment of the present disclosure, as the radii of rotation of the jaw 101 and the jaw 102 increase, an effect of increasing a yaw motion range in which a normal opening/closing actuation motion is performed may be obtained.

Next, components associated with rotation of the pulley 121 will be described.

The pulley 123 and the pulley 124 function as end tool second jaw pitch main pulleys. That is, the pulley 123 and the pulley 124 function as main rotation pulleys for a pitch motion of the second jaw 102. Here, the wire 306, which is a second jaw wire, is wound around the pulley 123, and the wire 302, which is another second jaw wire, is wound around the pulley 124.

The pulley 125 and the pulley 126 function as end tool second jaw sub-pulleys. That is, the pulley 125 and the pulley 126 function as sub-rotation pulleys for a pitch motion of the second jaw 102. Here, the wire 306, which is a second jaw wire, is wound around the pulley 125, and the wire 302, which is another second jaw wire, is wound around the pulley 126.

On one side of the pulley 121, the pulley 123 and the pulley 124 are arranged to face each other. Here, the pulley 123 and the pulley 124 are formed to be rotatable independently of each other around the rotation shaft 143, which is an end tool pitch rotation shaft. In addition, the pulley 125 and the pulley 126 are arranged on one sides of the pulley 123 and the pulley 124, respectively, to face each other. Here, the pulley 125 and the pulley 123 are formed to be rotatable independently of each other around the rotation shaft 144, which is an end tool pitch auxiliary rotation shaft. Here, the drawings illustrate that the pulley 123, the pulley 125, the pulley 124, and the pulley 126 are all formed to be rotatable around the Y-axis direction, but an embodiment of the present disclosure is not limited thereto, and the rotation shafts of the respective pulleys may be formed in various directions suitable for their configurations.

The wire 306, which is a second jaw wire, is wound to sequentially come into contact with at least portions of the pulley 125, the pulley 123, and the pulley 121. In addition, the wire 302 connected to the wire 306 by the fastening member 326 is wound to sequentially come into contact with at least portions of the pulley 121, the pulley 122, the pulley 124, and the pulley 126.

In other words, the wire 306 and the wire 302, which are second jaw wires, are wound to sequentially come into contact with at least portions of the pulley 125, the pulley 123, the pulley 121, the pulley 122, the pulley 124, and the pulley 126, and are formed to move along the above pulleys while rotating the above pulleys.

Accordingly, when the wire 306 is pulled in the direction of an arrow 306 of FIG. 9, the fastening member 326 to which the wire 306 is coupled and the pulley 121 coupled to the fastening member 326 rotate in the direction of the arrow R of FIG. 9. On the contrary, when the wire 302 is pulled in the direction of an arrow 302 of FIG. 9, the fastening member 326 to which the wire 302 is coupled and the pulley 121 coupled to the fastening member 326 rotate in the direction of the arrow L of FIG. 9.

Hereinafter, a pitch motion of the present disclosure will be described in more detail.

First, for a pitch motion, on the side of the end tool 100, the pulley 113, the pulley 114, the pulley 123, and the pulley 124, which are end tool jaw pitch main pulleys, are formed to be rotatable around the rotation shaft 143. Meanwhile, on the side of the proximal end 105 of the end tool jaw pitch main pulleys, the pulley 115, the pulley 116, the pulley 125, and the pulley 126, which are end tool jaw pitch sub-pulleys, are formed to be rotatable around the rotation shaft 144.

In addition, with respect to a plane (i.e., an XY plane) perpendicular to the rotation shaft 141 and including the rotation shaft 143, the wire 301 and the wire 305, which are two strands of the first jaw wire, are located on the same side with respect to the XY plane. That is, the wire 301 and the wire 305 are formed to pass through the lower sides of the pulley 114 and the pulley 113, which are end tool jaw pitch main pulleys, and the upper sides of the pulley 115 and pulley 116, which are end tool jaw pitch sub-pulleys.

Similarly, the wire 302 and the wire 306, which are two strands of the second jaw wire, are located on the same side with respect to the XY plane. That is, the wire 302 and the wire 306 are formed to pass through the upper sides of the pulley 123 and the pulley 124, which are end tool jaw pitch main pulleys, and the lower sides of the pulley 125 and pulley 126, which are end tool jaw pitch sub-pulleys.

In addition, for the wire 301 and the wire 305 that are two strands of the first jaw wire, when the wire 301 is pulled in the direction of the arrow 301 of FIG. 9, and simultaneously the wire 305 is pulled in the direction of the arrow 305 of FIG. 9 (i.e., when both strands of the first jaw wire are pulled in the same direction), the wire 301 and the wire 305 are wound around lower portions of the pulley 113 and the pulley 114 that are rotatable around the rotation shaft 143 that is an end tool pitch rotation shaft as illustrated in FIG. 8, thus, the pulley 111 to which the wire 301 and the wire 305 are fixedly coupled, and the end tool hub 106 to which the pulley 111 is coupled rotate together counterclockwise around the rotation shaft 143, and accordingly, the end tool 100 rotates downward to perform a pitch motion. At this time, because the second jaw 102 and the wire 302 and the wire 306 both fixedly coupled to the second jaw 102 are wound around upper portions of the pulley 123 and the pulley 124 that are rotatable around the rotation shaft 143, the wire 302 and the wire 306 are unwound in the opposite directions of the arrows 302 and 306, respectively.

On the contrary, for the wire 302 and the wire 306 that are two strands of the second jaw wire, when the wire 302 is pulled in the direction of the arrow 302 of FIG. 9, and simultaneously the wire 306 is pulled in the direction of the arrow 306 of FIG. 9 (i.e., when both strands of the second jaw wire are pulled in the same direction), the wire 302 and the wire 302 are wound above the pulley 123 and the pulley 124 that are rotatable around the rotation shaft 143 that is the end tool pitch rotation shaft as illustrated in FIG. 8, thus, the pulley 121 to which the wire 302 and the wire 306 are fixedly coupled, and the end tool hub 106 to which the pulley 121 is coupled are rotated together clockwise around the rotation shaft 143, and accordingly, the end tool 100 rotates upward to perform a pitch motion. At this time, because the first jaw 101 and the wire 301 and the wire 305 both fixedly coupled to the first jaw 101 are wound around lower portions of the pulley 113 and the pulley 114 that are rotatable around the rotation shaft 143, the wire 302 and the wire 306 are moved in the opposite directions of the arrows 301 and 305, respectively.

In other words, it may also be described that, when pitch rotation of the end tool 100 is performed, both strands of each jaw wire move simultaneously in the same direction.

In addition, the end tool 100 of the multi-joint surgical instrument 30 of the present disclosure may further include the pulley 131, which is an end tool pitch pulley, the driving part 200 may further include the pulley 231, which is a driving part pitch pulley, and the power transmission part 300 further include the wire 303 and the wire 304 that are pitch wires. In detail, the pulley 131 of the end tool 100 is rotatable around the rotation shaft 143, which is an end tool pitch rotation shaft, and may be formed with the end tool hub 106 as one body (or to be fixedly coupled to the end tool hub 106). In addition, the wire 303 and the wire 304 may serve to connect the pulley 131 of the end tool 100 to the pulley 231 of the driving part 200.

Thus, when the pulley 231 of the driving part 200 rotates, the rotation of the pulley 231 is transferred to the pulley 131 of the end tool 100 through the wire 303 and the wire 304 such that the pulley 131 rotates together, and accordingly, the end tool 100 rotates to perform a pitch motion.

The multi-joint surgical instrument 30 according to an embodiment of the present disclosure includes the pulley 131 of the end tool 100, the pulley 231 of the driving part 200, and the wire 303 and the wire 304 of the power transmission part 300, allowing a driving force of a pitch motion of the driving part 200 to be more completely transmitted to the end tool 100, thereby improving the operational reliability.

Here, the diameter of each of the pulley 113, the pulley 114, the pulley 123, and the pulley 124, which are end tool jaw pitch main pulleys, may be equal to or different from the diameter of the pulley 131, which is an end tool pitch pulley. Here, the ratio of the diameter of the end tool jaw pitch main pulley to the diameter of the end tool pitch pulley may be equal to the ratio of the diameter of a driving part relay pulley of the driving part 200, which will be described below, to the diameter of the driving part pitch pulley. This will be described in detail below.

Hereinafter, the driving part 200 of the multi-joint surgical instrument 30 of FIG. 6 will be described in more detail.

Referring to FIGS. 10 to 16, the driving part 200 of the multi-joint surgical instrument 30 according to an embodiment of the present disclosure may include the pulley 211, the pulley 212, a pulley 213, a pulley 214, a pulley 215, a pulley 216, a pulley 217, a pulley 218, a pulley 219, and a pulley 220, which are associated with a rotational motion of the first jaw 101. In addition, the driving part 200 may include the pulley 221, the pulley 222, a pulley 223, a pulley 224, a pulley 225, a pulley 226, a pulley 227, a pulley 228, a pulley 229, and a pulley 230, which are associated with a rotational motion of the second jaw 102.

Here, although the drawings illustrate that the pulleys facing each other are arranged in parallel with each other, an embodiment of the present disclosure is not limited thereto, and the pulleys may be formed in various positions and sizes suitable for the configuration of the driving part.

In addition, the driving part 200 of the multi-joint surgical instrument 30 according to an embodiment of the present disclosure may further include the pulley 231 serving as a driving part pitch pulley, and a pitch-yaw connector 232 connecting the pulley 231 to the above-described driving part jaw pulleys.

In addition, the driving part 200 of an embodiment of the present disclosure may include a rotation shaft 241, a rotation shaft 242, a rotation shaft 243, a rotation shaft 244, a rotation shaft 245, and a rotation shaft 246. Here, the rotation shaft 241 may function as a driving part first jaw rotation shaft, and the rotation shaft 242 may function as a driving part second jaw rotation shaft. In addition, the rotation shaft 243 may function as a driving part pitch rotation shaft, and the rotation shaft 244 may function as a driving part roll rotation shaft. In addition, the rotation shaft 245 may function as a driving part first jaw auxiliary rotation shaft, and the rotation shaft 246 may function as a driving part second jaw auxiliary rotation shaft. One or more pulleys may be fit into each of the rotation shafts 241, 242, 243, 244, 245, and 246, and this will be described in detail below.

In addition, the driving part 200 of an embodiment of the present disclosure may include a motor coupling part 251, a motor coupling part 252, a motor coupling part 253, and a motor coupling part 254. Here, the motor coupling part 251 may function as a first jaw driving motor coupling part, the motor coupling part 252 may function as a second jaw driving motor coupling part, the motor coupling part 253 may function as a pitch driving motor coupling part, and the motor coupling part 254 may function as a roll driving motor coupling part. Here, each of the motor coupling parts 251, 252, 253, and 254 may be formed in the form of a rotatable flat plate, and may have formed thereon one or more coupling holes to which a motor (not shown) may be coupled.

The motor coupling parts 251, 252, 253, and 254 of the driving part 200 as described above are coupled to motors (not shown) formed in the robotic arm units 21, 22, and 23, respectively, such that the driving part 200 is operated by driving of the motors (not shown).

In addition, the driving part 200 of an embodiment of the present disclosure may include a gear 261, a gear 262, a gear 263, and a gear 264. Here, the gear 261 and the gear 262 may function as pitch driving gears, and the gear 263 and the gear 264 may function as roll driving gears. Hereinafter, each component will be described in more detail.

The pulley 211 and the pulley 212 may function as driving part first jaw pulleys, the pulley 221 and the pulley 222 may function as driving part second jaw pulleys, and these components may be collectively referred to as driving part jaw pulleys.

Here, the drawings illustrate that the pulley 211 is associated with a rotational motion of the first jaw 101 of the end tool 100, and the pulley 221 is associated with a rotational motion of the second jaw 102 of the end tool 100, but an embodiment of the present disclosure is not limited thereto. For example, one group of pulleys within the driving part may be associated with a yaw motion, and another group of pulleys may be associated with an actuation motion. Thus, the pulley 211 and the pulley 212 may be collectively referred to as driving part driving pulleys. In addition, for other pulleys to be described below, one group of pulleys may be associated with a yaw motion, and another group of pulleys may be associated with an actuation motion.

The pulley 213 and the pulley 214 may function as driving part first jaw auxiliary pulleys, the pulley 223 and the pulley 224 may function as driving part second jaw auxiliary pulleys, and these components may be collectively referred to as driving part auxiliary pulleys.

The pulley 215 and the pulley 216 may function as driving part first jaw first relay pulleys, the pulley 217 and the pulley 218 may function as driving part first jaw second relay pulleys, and these components may be collectively referred to as driving part first jaw relay pulleys. Meanwhile, the pulley 225 and the pulley 226 may function as driving part second jaw first relay pulleys, the pulley 227 and the pulley 228 may function as driving part second jaw second relay pulleys, and these components may be collectively referred to as driving part second jaw relay pulleys. In addition, the pulley 215, the pulley 216, the pulley 225, and the pulley 226 may be collectively referred to as driving part first relay pulleys, and the pulley 217, the pulley 218, the pulley 227, and the pulley 228 may be collectively referred to as driving part second relay pulleys. Furthermore, the pulley 215, the pulley 216, the pulley 217, the pulley 218, the pulley 225, the pulley 226, the pulley 227, and the pulley 228 may be collectively referred to as driving part relay pulleys.

Here, the drawings illustrate that two pulleys are paired to serve as driving part relay pulleys for each jaw, but an embodiment of the present disclosure is not limited thereto. For example, it is illustrated that the pulley 215, which is a driving part first jaw first relay pulley, and the pulley 217, which is a driving part first jaw second relay pulley, are formed as a pair, and the wire 301 sequentially passes through the pulley 215 and the pulley 217. However, three or more pulleys, rather than two pulleys, may serve as driving part first jaw relay pulleys.

Meanwhile, the pulley 219 and the pulley 220 may function as driving part first jaw satellite pulleys, the pulley 229 and the pulley 230 may function as driving part second jaw satellite pulleys, and these components may be collectively referred to as driving part satellite pulleys.

A plurality of rotation shafts including the rotation shaft 241, the rotation shaft 242, the rotation shaft 243, the rotation shaft 244, the rotation shaft 245, and the rotation shaft 246 may be formed on a first surface of a base plate 201. In addition, a plurality of relay pulleys 202 may be formed on the first surface of the base plate 201, to serve to redirect, toward the pulley 231, the wires 301, 302, 303, 304, 305, and 306 that have entered the driving part 200 through the connection part 310.

In addition, the connection part 310 in the form of a shaft may be coupled to a second surface of the base plate 201 opposite to the first surface, and the motor coupling part 251, the motor coupling part 252, the motor coupling part 253, and the motor coupling part 254, to which the motors (not shown) for driving the pulleys are coupled, may be formed on the second surface.

Here, each rotation shaft and each motor coupling part may be directly connected or indirectly connected via a gear.

For example, by directly coupling the motor coupling part 251, which is a first jaw driving motor coupling part, to the rotation shaft 241, which is a driving part first jaw rotation shaft, when the motor coupling part 251 coupled to a first jaw driving motor (not shown) rotates, the rotation shaft 241 directly coupled to the motor coupling part 251 may rotates together. Similarly, by directly coupling the motor coupling part 252, which is a second jaw driving motor coupling part, to the rotation shaft 242, which is a driving part second jaw rotation shaft, when the motor coupling part 252 coupled to a second jaw driving motor (not shown) rotates, the rotation shaft 242 directly coupled to the motor coupling part 252 may rotates together.

As another example, when viewed from a plane perpendicular to the rotation shaft 243, the motor coupling part 253, which is a pitch driving motor coupling part, and the rotation shaft 243, which is a driving part pitch rotation shaft, may be arranged to be spaced apart from each other by a certain extent. In addition, the motor coupling part 253 and the rotation shaft 243 may be connected to each other by the gear 261 and the gear 262, which are pitch driving gears.

Similarly, when viewed from a plane perpendicular to the rotation shaft 244, the motor coupling part 254, which is a roll driving motor coupling part, and the rotation shaft 244, which is a driving part roll rotation shaft, may be arranged to be spaced apart from each other by a certain extent. In addition, the motor coupling part 254 and the rotation shaft 244 may be connected to each other by the gear 263 and the gear 264, which are roll driving gears.

As such, some motor coupling parts are configured to be directly connected to the rotation shafts, respectively, and the other motor coupling parts are configured to be indirectly connected to the rotation shafts, respectively, because the coupling position and direction between the multi-joint surgical instrument 30 and the slave robot 20 need to be considered. That is, the rotation shaft that is not affected by the coupling position with the slave robot 20 may be directly connected to the motor coupling part, whereas the rotation shaft that may cause interference with the coupling position with the slave robot 20 may be indirectly connected to the motor coupling part.

The drawings illustrate that the motor coupling part 251 and the motor coupling part 252 are directly connected to the rotation shafts, and the motor coupling part 253 and the motor coupling part 254 are indirectly connected to the rotation shafts through the gears, but an embodiment of the present disclosure is not limited thereto, and various configurations are possible according to the coupling position and direction with the slave robot 20.

The pulley 211 and the pulley 212, which are driving part first jaw pulleys, may be coupled to the rotation shaft 241, which is a driving part first jaw rotation shaft. Here, the pulley 211 and the pulley 212 may be formed to rotate together with the rotation shaft 241.

In addition, the rotation shaft 245, which is a driving part first jaw auxiliary rotation shaft, may be arranged in a region adjacent to the rotation shaft 241. The pulley 213 and the pulley 214, which are driving part first jaw auxiliary pulleys, may be coupled to the rotation shaft 245. Here, the pulley 213 and the pulley 214 may be formed to be rotatable around the rotation shaft 245.

Here, the drawings illustrate that two pulleys 211 and 212 serve as driving part first jaw pulleys, the wire 301 is coupled to one pulley 211, and the wire 305 is coupled to the other pulley 212. However, an embodiment of the present disclosure is not limited thereto, and only one pulley may serve as a driving part first jaw pulley, and both the wire 301 and the wire 305 may be coupled to this pulley.

As described above, the rotation shaft 241 is coupled to the first jaw driving motor (not shown) by the motor coupling part 251, and thus, when the first jaw driving motor (not shown) rotates for driving of the first jaw 101, the pulley 211 and the pulley 212, which are driving part first jaw pulleys, rotate together with the rotation shaft 241, such that the wire 301 and the wire 305, which are first jaw wires, are pulled or released.

The pulley 221 and the pulley 222, which are driving part second jaw rotation shafts, may be coupled to the rotation shaft 242 that is a driving part second jaw pulley. Here, the pulley 221 and the pulley 222 may be formed to rotate together with the rotation shaft 242.

In addition, the rotation shaft 246, which is a driving part second jaw auxiliary rotation shaft, may be arranged in a region adjacent to the rotation shaft 242. The pulley 223 and the pulley 224, which are driving part second jaw auxiliary pulleys, may be coupled to the rotation shaft 245. Here, the pulley 223 and the pulley 224 may be formed to be rotatable around the rotation shaft 246.

Here, the drawings illustrate that two pulleys 221 and 222 serve as driving part second jaw pulleys, the wire 302 is coupled to one pulley 221, and the wire 306 is coupled to the other pulley 222. However, an embodiment of the present disclosure is not limited thereto, and only one pulley may serve as a driving part second jaw pulley, and both the wire 302 and the wire 306 may be coupled to this pulley.

As described above, the rotation shaft 242 is coupled to the second jaw driving motor (not shown) by the motor coupling part 252, and thus, when the second jaw driving motor (not shown) rotates for driving of the second jaw 102, the pulley 221 and the pulley 222, which are driving part second jaw pulleys, rotate together with the rotation shaft 242, such that the wire 302 and the wire 306, which are second jaw wires, are pulled or released.

The pulley 231, which is a driving part pitch pulley, may be coupled to the rotation shaft 243 that is a driving part pitch rotation shaft. Here, the pulley 231 may be formed to rotate together with the rotation shaft 243.

As described above, the rotation shaft 243 is coupled to a pitch driving motor (not shown) by the motor coupling part 253, and thus, when the pitch driving motor (not shown) rotates for a pitch motion, the wire 303 and the wire 304, which are pitch wires, are pulled or released as the pulley 231, which is a driving part pitch pulley, rotates together with the rotation shaft 243.

In addition, the pulley 215, the pulley 216, the pulley 217, the pulley 218, the pulley 225, the pulley 226, the pulley 227, and the pulley 228, which are driving part relay pulleys, may be inserted through the rotation shaft 243 to be rotatable around the rotation shaft 243. Here, the pulley 215, the pulley 216, the pulley 217, and the pulley 218, which are driving part first jaw relay pulleys, may be arranged on one surface side of the pulley 231, which is a pitch pulley, and the pulley 225, the pulley 226, the pulley 227, and the pulley 228, which are driving part second jaw relay pulleys, may be arranged on the other surface side of the pulley 231.

In other words, the pulley 225 and the pulley 226, which are driving part second jaw first relay pulleys, the pulley 227 and the pulley 228, which are driving part second jaw second relay pulleys, the pulley 231, which is a driving part pitch pulley, and the pulley 217 and the pulley 218, which are driving part first jaw second relay pulleys, and the pulley 215 and the pulley 216, which are driving part first jaw first relay pulleys, are sequentially stacked along the rotation shaft 243.

In addition, the pitch-yaw connector 232 may be coupled to the rotation shaft 243. The pitch-yaw connector 232 may be formed to rigidly connect the pulley 231, which is a driving part pitch pulley, to the pulley 219, the pulley 220, the pulley 229, and the pulley 230, which are driving part satellite pulleys, to allow the driving part satellite pulleys to revolve around the rotation shaft 243 when the pulley 231 rotates. This will be described in more detail below.

Here, the pitch-yaw connector 232 may be formed to rotate together with the rotation shaft 243. That is, the pulley 231 and the pitch-yaw connector 232 may be coupled to the rotation shaft 243, to rotate together with the rotation shaft 243.

Here, the pitch-yaw connector 232 may be described as being formed in a substantially Y-shape as illustrated in FIG. 12, or may be described as being formed in a shape in which at least two extension parts 232a and 232b are formed to extend from the center thereof. In addition, a driving part first jaw satellite pulley central shaft 233 and a driving part second jaw satellite pulley central shaft 234 may be formed at ends of the extension parts 232a and 232b, respectively.

In addition, the pulley 219 and the pulley 220, which are driving part first jaw satellite pulleys, may be coupled to the driving part first jaw satellite pulley central shaft 233, and the pulley 229 and the pulley 230, which are driving part second jaw satellite pulleys, may be coupled to the driving part second jaw satellite pulley central shaft 234.

Accordingly, when the pulley 231, which is a driving part pitch pulley, rotates together with the rotation shaft 243, the pulley 219, the pulley 220, the pulley 229, and the pulley 230, which are driving part satellite pulleys, revolve around the rotation shaft 243. In other words, it may also be described that that the driving part first jaw satellite pulley central shaft 233 and the driving part second jaw satellite pulley central shaft 234 rotate around the rotation shaft 243 while maintaining a constant distance from the rotation shaft 243, in a state in which the driving part first jaw satellite pulley central shaft 233 and the driving part second jaw satellite pulley central shaft 234 are spaced apart from the rotation shaft 243 by a certain extent.

That is, the driving part satellite pulley is formed to be movable relative to the driving part relay pulley and the rotation shaft 243 such that relative positions of the driving part satellite pulley with respect to the driving part relay pulley and the rotation shaft 243 may be changed. On the contrary, the relative positions of the driving part pitch pulley and the driving part relay pulley remain constant.

In addition, when the pulley 231, which is a driving part pitch pulley, rotates around the rotation shaft 243, the pulley 219, the pulley 220, the pulley 229, and the pulley 230, which are driving part satellite pulleys, move relative to the pulley 231, which is a driving part pitch pulley, such that the overall lengths of the wire 301, the wire 302, the wire 305, and the wire 306, which are jaw wires, within the driving part 200 are changed.

The wire 301, which is a first jaw wire, is connected to the end tool 100 through the connection part 310 after being sequentially wound to come into contact with at least portions of the pulley 211, the pulley 213, the pulley 215, the pulley 219, and the pulley 217, in a state in which one end of the wire 301 is coupled to the pulley 211 by the first jaw wire-driving part fastening member (not shown).

In other words, the wire 301, which is a first jaw wire, is connected to the end tool 100 through the connection part 310 after being sequentially passing through the driving part first jaw pulley 211, the driving part first jaw auxiliary pulley 213, the driving part first jaw first relay pulley 215, the driving part first jaw satellite pulley 219, and the driving part first jaw second relay pulley 217.

In other words, the wire 301, which is a first jaw wire, enters the driving part 200 after passing through the end tool 100 and the connection part 310, and then is fixedly coupled to the pulley 211, which is a driving part first jaw pulley, after being sequentially wound around the pulley 217, the pulley 219, the pulley 215, and the pulley 213.

In addition, the wire 305, which is a first jaw wire, is connected to the end tool 100 through the connection part 310 after being sequentially wound to come into contact with at least portions of the pulley 212, the pulley 214, the pulley 216, the pulley 220, and the pulley 218, in a state in which one end of the wire 305 is coupled to the pulley 212 by the first jaw wire-driving part fastening member (not shown).

The wire 302, which is a second jaw wire, is connected to the end tool 100 through the connection part 310 after being sequentially wound to come into contact with at least portions of the pulley 221, the pulley 223, the pulley 225, the pulley 229, and the pulley 227, in a state in which one end of the wire 302 is coupled to the pulley 221 by the second jaw wire-driving part fastening member (not shown).

In addition, the wire 306, which is a second jaw wire, is connected to the end tool 100 through the connection part 310 after being sequentially wound to come into contact with at least portions of the pulley 222, the pulley 224, the pulley 226, the pulley 230, and the pulley 228, in a state in which one end of the wire 306 is coupled to the pulley 222 by the second jaw wire-driving part fastening member (not shown).

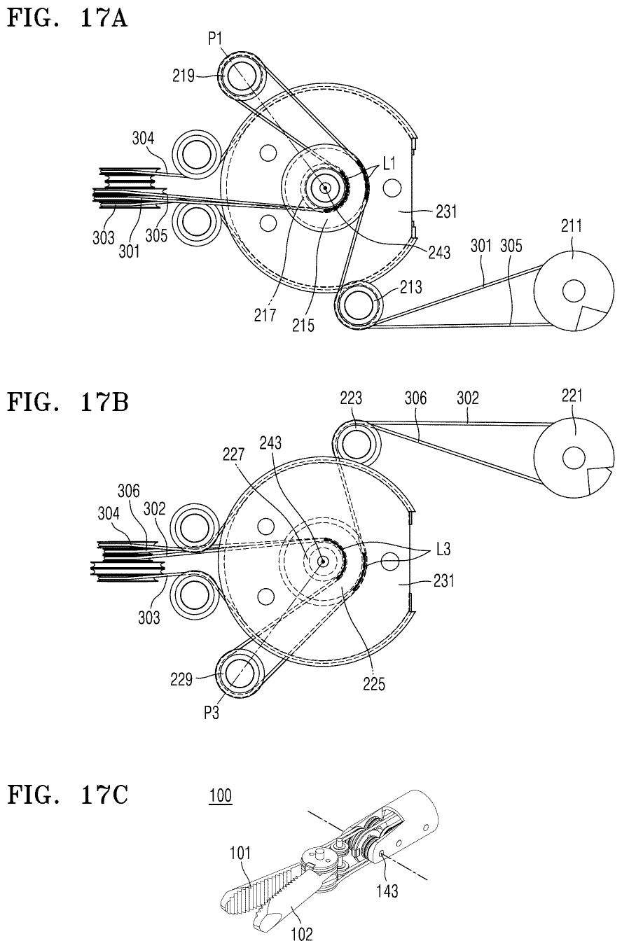

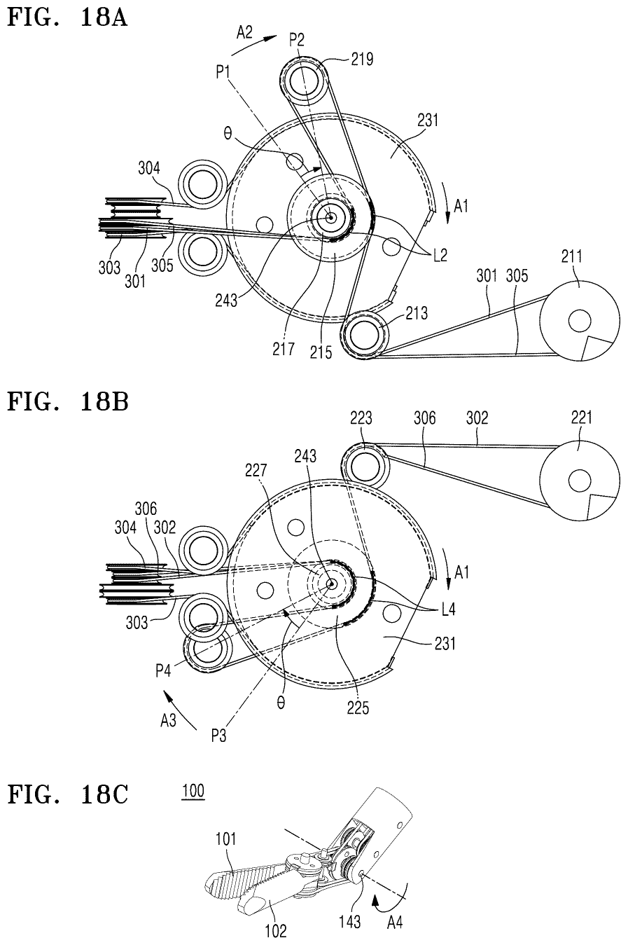

FIGS. 17 and 18 are diagrams illustrating a pitch motion of the multi-joint surgical instrument illustrated in FIG. 6. Here, for convenience of description, only pulleys and wires associated with rotation of the first jaw are illustrated in (a) of FIG. 17 and (a) of FIG. 18, and only pulleys and wires associated with rotation of the second jaw are illustrated in (b) of FIG. 17 and (b) of FIG. 18. In addition, (c) of FIG. 17 and (c) of FIG. 18 illustrate a pitch motion of the end tool according to a pitch motion of the driving part.

Here, in the multi-joint surgical instrument 30 according to an embodiment of the present disclosure, when the driving part satellite pulley moves relative to the driving part relay pulley, the overall length of the jaw wires within the driving part 200 is changed, and thus, a pitch motion of the end tool 100 is performed. In particular, in the multi-joint surgical instrument 30 according to an embodiment of the present disclosure, when the driving part pitch pulley rotates, the driving part satellite pulley revolves around the (common) rotation shaft of the driving part relay pulley and the driving part pitch pulley such that path length of the jaw wire wound around the driving part relay pulley is changed, and thus, a pitch motion of the end tool is performed.

In detail, when motion compensation for the pitch motion is not separately performed in the driving part, the pitch motion itself cannot be performed in the end tool.

Meanwhile, in order for the end tool to perform a pitch motion, the wire 301 and the wire 305 need to be further wound around the pulley 113 by ΔS, and the wire 302 and the wire 306 need to be further unwound from the pulley 114 by ΔS. However, when such compensation is not performed in the driving part, the pitch motion itself cannot be performed in the end tool.

In order to perform motion compensation for the pitch motion as described above, in the multi-joint surgical instrument 30 according to an embodiment of the present disclosure, the driving part pitch pulleys rotate while the driving part satellite pulleys revolve, such that the jaw wires are wound around or released from the driving part relay pulleys, which allows the movement of the jaw wires to be compensated for by the rotation of the driving part pitch pulleys.

In other words, when the pulley 231, which is a driving part pitch pulley, rotates together with the rotation shaft 243, the driving part satellite pulleys revolve around the rotation shaft 243. In addition, as the driving part satellite pulleys revolve around the rotation shaft 243, the length by which the jaw wire is wound around the driving part relay pulley is changed. That is, the jaw wire wound on the side of the end tool 100 due to rotation of the pulley 231 is released by the same amount on the side of the driving part 200, and the jaw wire unwound on the side of the end tool 100 is wound by the same amount on the side of the driving part 200, such that the pitch motion does not affect the yaw motion.

In other words, when the end tool performs the pitch motion by the rotation of the driving part pitch pulley, the jaw wire (responsible for a yaw motion and an actuation motion) is also moved by the pitch motion. That is, as the pitch rotation is performed around the rotation shaft 143 of the end tool 100, both strands of the jaw wire coupled to one jaw are pulled, and both strands thereof coupled to the other jaw are released. Accordingly, it may also be described that, in the present disclosure, in order to compensate for the movement of the jaw wire, when the pitch motion of the end tool is performed, the overall length of the jaw wire within the driving part is changed while the driving part satellite pulley moves relative to the driving part relay pulley, such that the jaw wire is released (or pulled) on the side of the end tool as much as the jaw wire is pulled (or released) on the side of the driving part, thereby compensating for the movement of the jaw wire when the pitch motion of the end tool is performed.

Hereinafter, the pitch motion will be described in more detail.

When the pulley 231, which is a driving part pitch pulley, rotates in the direction of an arrow A1 (i.e., in the clockwise direction in the drawing) in order to perform the pitch motion, the pitch-yaw connector (see 232 of FIG. 10) rotates in the direction of the arrow A1 together with the pulley 231, and accordingly, the pulley 219 and the pulley 220, which are driving part satellite pulleys fixedly coupled to the pitch-yaw connector (see 232 of FIG. 10), revolve as a whole in the direction of an arrow A2 of (a) of FIG. 18 (i.e., in the clockwise direction in the drawing) around the rotation shaft 243 by θ. That is, when the pulley 231 rotates, the pulley 219 and the pulley 220 rotate by θ from the position of P1 of (a) of FIG. 17 to the position of P2 of (a) of FIG. 18. In other words, it may be described that, when the driving part pitch pulley rotates, the driving part satellite pulley moves in conjunction with the driving part pitch pulley.

At the same time, when the pulley 231, which is a driving part pitch pulley, rotates in the direction of the arrow A1 (i.e., in the clockwise direction in the drawing), the pitch-yaw connector (see 232 of FIG. 10) rotates in the direction of the arrow A1 together with the pulley 231, and accordingly, the pulley 229 and the pulley 230, which are driving part satellite pulleys fixedly coupled to the pitch-yaw connector (see 232 of FIG. 10), revolve as a whole in the direction of an arrow A3 of (b) of FIG. 18 (i.e., in the clockwise direction in the drawing) around the rotation shaft 243 by θ. That is, when the pulley 231 rotates, the pulley 229 and the pulley 230 rotate by θ from the position of P3 of (b) of FIG. 17 to the position of P4 of (b) of FIG. 18. In other words, it may be described that, when the driving part pitch pulley rotates, the driving part satellite pulley moves in conjunction with the driving part pitch pulley.

In addition, at this time, the positions of the pulley 215, the pulley 216, the pulley 217, the pulley 218, the pulley 225, the pulley 226, the pulley 227, and the pulley 228, which are driving part relay pulleys coupled to the rotation shaft 243, are not changed. That is, the relative positions of the pulley 211, which is a driving part jaw pulley, the pulley 231, which is a driving part pitch pulley, and the pulley 215, the pulley 216, the pulley 217, and the pulley 218, which are driving part relay pulleys, remain constant. Similarly, the relative positions of the pulley 221, which is a driving part jaw pulley, the pulley 231, which is a driving part pitch pulley, and the pulley 225, the pulley 226, the pulley 227, and the pulley 228, which are driving part relay pulleys, remain constant.

In addition, as described above, the relative position of the driving part satellite pulley with respect to the driving part relay pulley is changed as the driving part satellite pulley revolves, and accordingly, the length of each wire wound around the driving part relay pulley, that is, the path length, is changed. Here, the driving part relay pulleys include the pulley 215, which is a driving part first jaw first relay pulley, and the pulley 217, which is a driving part first jaw second relay pulley, and thus, the path length means the sum of the length by which the wire 301 is wound around the pulley 215, and the length by which the wire 301 is wound around the pulley 217 (or, the sum of the length by which the wire 305 is wound around the pulley 216, and the length by which the wire 305 is wound on the pulley 218).

That is, as compared to a path length L1 by which the wire 301 and the wire 305, which are first jaw wires, are wound around the driving part relay pulleys at the positions in (a) of FIG. 17, a path length L2 by which the first jaw wires are wound around the driving part relay pulleys at the positions in (a) of FIG. 18 decreases, and the first jaw wires are further released on the side of the driving part 200 as much as the path length decreases (L1−L2). That is, the overall length of the wire 301 and the wire 305, which are first jaw wires, within the driving part 200 decreases. In addition, as the overall length of the first jaw wires within the driving part 200 decreases, the overall length of the first jaw wires within the end tool 100 increases as much as the first jaw wires are unwound.

In contrast, when the pulley 231, which is a driving part pitch pulley, rotates in the direction of the arrow A1, as compared to a path length L3 by which the wire 302 and the wire 306, which are second jaw wires, are wound around the driving part relay pulleys at the positions in (b) of FIG. 17, a path length L4 by which the second jaw wires are wound around the driving part relay pulleys at the positions in (b) of FIG. 18 increases, and the second jaw wires are further pulled on the side of the driving part 200 as much as the path length increases (L4-L3). That is, the overall length of the wire 302 and the wire 306, which are second jaw wires, within the driving part 200 increases. In addition, as the overall length of the second jaw wires within the driving part 200 increases, the overall length of the second jaw wires within the end tool 100 decreases as much as the second jaw wires are pulled.

As such, when the pulley 231, which is a driving part pitch pulley, rotates in the direction of the arrow A1 for a pitch motion, the relative position of the driving part satellite pulley is changed as the driving part satellite pulley moves relative to the driving part pitch pulley and the driving part relay pulley. In addition, due to the relative movement of the driving part satellite pulley, the overall length of the first jaw wires within the driving part 200 decreases, and the overall length of the first jaw wires within the end tool 100 increases. At the same time, due to the relative movement of the driving part satellite pulley, the overall length of the second jaw wires within the driving part 200 increases, and the overall length of the second jaw wires within the end tool 100 decreases.

Accordingly, when viewed from the side of the end tool 100, when the pulley 231, which is a driving part pitch pulley, rotates in the direction of the arrow A1, the wire 301 and the wire 305, which are two strands of the first jaw wire, are released and the wire 302 and the wire 306, which are two strands of the second jaw wire, are pulled, such that the end tool 100 performs a pitch motion in the direction of an arrow A4 around the rotation shaft 143.

Here, the path length may be defined as a length of the jaw wire from a point at which the jaw wire enters the driving part first relay pulley, to a point at which the jaw wire exits from the driving part second relay pulley through the driving part satellite pulley. That is, the path length may be defined as a length of the wire 301, which is a jaw wire, from a point at which the jaw wire enters the pulley 215, which is a driving part first relay pulley, to a point at which the jaw wire exits from the pulley 217, which is a driving part second relay pulley, through the pulley 219 that is a driving part satellite pulley.

In other words, the path length may be defined as the length of the jaw wire from an initial contact point of the jaw wire with the driving part relay pulley to a final contact point of the jaw wire with the driving part relay pulley on the arrangement path of the jaw wire that connects the end tool jaw pulley to the driving part jaw pulley. That is, the path length may be defined as the length of the jaw wire from an initial contact point of the wire 301, which is a jaw wire, with the pulley 215, which is a driving part first relay pulley, to a final contact point of the wire 301 with the pulley 217, which is a driving part second relay pulley.

Meanwhile, as the above-described path length is changed as the driving part satellite pulley moves relative to the driving part relay pulley, the overall length of the jaw wire within the driving part 200 is also changed. In addition, as the overall length of the jaw wire within the driving part 200 is changed, the overall length of the jaw wire within the end tool 100 is also changed. However, it may be described that, because the overall length of the jaw wire within the end tool 100 also decreases (or increases) as much as the overall length of the jaw wire within the driving part 200 increases (decreases), the overall length of the jaw wire is not changed (assuming that elastic deformation or the like is not considered).

Accordingly, when the driving part pitch pulley rotates, the wire 301/wire 305, which are first jaw wires, are released on the side of the driving part 200 as much as the wire 301/wire 305, which are first jaw wires, are pulled on the side of the end tool 100, and as a result, a pitch motion is enabled.

In addition, as described above, the end tool 100 of the multi-joint surgical instrument 30 of the present disclosure may further include the pulley 131, which is an end tool pitch pulley, the driving part 200 may further include the pulley 231, which is a driving part pitch pulley, and the power transmission part 300 further include the wire 303 and the wire 304 that are pitch wires.

Thus, when the pulley 231, which is a driving part pitch pulley, rotates in the direction of the arrow A1, the wire 304 is wound around the pulley 231 and the wire 303 is released from the pulley 231, due to the rotation of the pulley 231. Accordingly, as the pulley 131, which is an end tool pitch pulley connected to the opposite side of the wire 303 and the wire 304, rotates in the direction of the arrow A2 around the rotation shaft 143, the pitch motion may be performed more surely and reliably.

Here, among the pulleys that rotates around the rotation shaft 143, which is an end tool pitch rotation shaft, the pulley 131, which is an end tool pitch pulley to be in contact with the wire 303 and the wire 304, which are pitch wires, may be formed to have a diameter different from those of the pulley 113, the pulley 114, the pulley 123, and the pulley 124, which are end tool jaw pitch main pulleys to be in contact with the wire 301, the wire 305, the wire 302, and the wire 306, which are jaw wires.

In this case, when the rotation shaft 143 rotates, the lengths by which the respective wires are wound around or unwound from the respective pulleys are different from each other. For example, when the diameter of the end tool pitch pulley is 60, the diameter of the end tool jaw pitch main pulley is 40, and the rotation shaft 143 rotates by 90°, the length by which the pitch wire is wound around the end tool pitch pulley may be 1.5π, whereas the length by which the jaw wire is wound around the end tool jaw pitch main pulley may be 1π.

From this perspective, the ‘length’ by which the wire is wound around or unwound from the pulley may be defined as a ‘rotation amount’. The rotation amount is a concept different from a rotation angle, and may be calculated as (diameter*rotation angle/360°*π).

In this case, because basically the pulley 231, which is a driving part pitch pulley, is directly connected to the pulley 131, which is an end tool pitch pulley, by the wire 303 and the wire 304, which are pitch wires, the rotation amount of the driving part pitch pulley is equal to that of the end tool pitch pulley. That is, the pitch wire is unwound from or wound around the end tool pitch pulley as much as the pitch wire is wound around or unwound from the driving part pitch pulley.

In addition, the relationship of (diameter of end tool pitch pulley:diameter of end tool jaw pitch main pulley)=(rotation amount of wire wound around end tool pitch pulley: rotation amount of wire wound around end tool jaw pitch main pulley) may be satisfied.

As described above, when, in the end tool 100, the length by the pitch wire is wound around the end tool pitch pulley is different from the length by which the jaw wire is wound around the end tool jaw pitch main pulley, in the driving part 200, the length by which the pitch wire is to be unwound needs to be different from the length by which the jaw wire is to be unwound, by the same proportion.

To this end, the relationship of (diameter of end tool pitch pulley:diameter of end tool jaw pitch main pulley)=(diameter of driving part pitch pulley:diameter of driving part relay pulley) may be satisfied.

For example, when the ratio of (diameter of end tool pitch pulley:diameter of end tool jaw pitch main pulley) is 6:4, the ratio of (diameter of driving part pitch pulley:diameter of driving part relay pulley) may also be 6:4. According to this ratio, the diameter of the driving part pitch pulley may be 90, and the diameter of the driving part relay pulley may be 60.

However, here, the driving part relay pulley may include two (or more) pulleys, i.e., the driving part first relay pulley and the driving part second relay pulley. In addition, the sum of the diameters of the driving part first relay pulley and the driving part second relay pulley may be defined as the diameter of the driving part relay pulley.

For example, when the diameter of the driving part relay pulley is 60, there are several possible combinations for (diameter of driving part first relay pulley, diameter of driving part second relay pulley), including (1φ, 5φ), (2φ, 4φ), (3φ, 3φ), (4φ, 2φ), and (5φ, 1φ). Here, the drawings illustrate that the diameter of the pulley 215, which is a driving part first relay pulley, is 4φ, and the diameter of the pulley 217, which is the driving part second relay pulley, is 2φ.

In addition, it may be described that (rotation amount of driving part first relay pulley+rotation amount of driving part second relay pulley) is proportional to the rotation amount of the driving part pitch pulley.

However, although the ratio of (diameter of end tool pitch pulley:diameter of end tool jaw pitch main pulley) is not exactly equal to the ratio of (diameter of driving part pitch pulley:diameter of driving part relay pulley), when the diameters of the pulleys are selected to make these ratios similar to each other, an objective of the present disclosure, which is to compensate for the movement of the jaw wire according to rotation of the driving part pitch pulley, may be achieved to some extent.

The final pitch motion process will be described again as follows.

Hereinafter, an example will be described in which the diameter of the end tool pitch pulley is 6φ, the diameter of the end tool jaw pitch main pulley is 4φ, the diameter of the driving part pitch pulley is 9φ, and the diameter of the driving part relay pulley is 6φ.

First, for a pitch motion, the pulley 231, which is a driving part pitch pulley of the driving part 200, rotates by 60° to wind the wire 304, which is a pitch wire, while unwinding the wire 303. At this time, the lengths by which the wire 303/wire 304 are respectively wound and unwound is 1.5π.

Accordingly, as the wire 304 is pulled by 1.5π and the wire 303 is released by 1.5π in the end tool 100, the pulley 131, which is an end tool pitch pulley, rotates by 90° corresponding to 1.5π.

In addition, when the pulley 131 pitch-rotates around the rotation shaft 143, the jaws 101 and 102 and the pulley 111/pulley 112 also pitch-rotate around the rotation shaft 143. Accordingly, both the wire 301 and the wire 305, which are first jaw wires coupled to the pulley 111, are pulled, and both the wire 302 and the wire 306, which are second jaw wires coupled to the pulley 121, are released. At this time, the angles by which the end tool pitch pulley and the end tool jaw pitch main pulley rotate are equal to each other, i.e., 90°, and thus, the lengths by which the jaw wires are wound around or unwound from the end tool jaw pitch main pulley are 1π.

In addition, because the pulley 231 and the pulley 219/pulley 220 are rigidly connected to each other by the pitch-yaw connector 232, when the pulley 231 rotates by 60° around the rotation shaft 243, the pulley 219/pulley 220 revolve by 60° around the rotation shaft 243.

In addition, as described above, as the pulley 219/pulley 220 revolve, the jaw wires are wound around or unwound from the pulley 215 and the pulley 216 of which the sum of the diameters is 6φ, by 1π corresponding to a revolution angle of 60°. That is, the wire 301 and the wire 305, which are first jaw wires, are released as a whole, and the wire 302 and the wire 306, which are second jaw wires, are pulled as a whole.

In other words, the overall path length by which the wire 301 and the wire 305 are wound around the pulley 215, the pulley 216, the pulley 217, and the pulley 218, which are driving part first jaw relay pulleys, decreases, and the wire 301 and the wire 305 are unwound as much as the path length decreases. In addition, the overall path length by which the wire 302 and the wire 306 are wound around the pulley 225, the pulley 226, the pulley 227, and the pulley 228, which are driving part second jaw relay pulleys, increases, and the wire 302 and the wire 306 are pulled as much as the path length increases.

That is, the wire 301 and the wire 305, which are first jaw wires, are released on the side of the driving part 200 as much as the wire 301 and the wire 305 are pulled on the side of the end tool 100, and thus, the movement of the jaw wires due to the pitch motion is compensated for. Similarly, the wire 302 and the wire 306, which are second jaw wires, are released on the side of the driving part 200 as much as the wire 302 and the wire 306 are pulled on the side of the end tool 100, and thus, the movement of the jaw wires due to the pitch motion is compensated for.

Accordingly, by releasing (or pulling) the jaw wires on the side of the driving part 200 by a length equal to the length by which the jaw wires are wound around (or unwound from) on the side of the end tool 100 according to the pitch motion, the pitch motion may be performed independently without affecting rotation of the jaw around the yaw shaft.

That is, when the driving part pitch pulley and the driving part satellite pulley are rigidly connected to each other, and the driving part pitch pulley rotates around the rotation shaft 243, the path length of the jaw wire wound around the driving part relay pulley is changed as the driving part satellite pulley revolves around the rotation shaft 243. In addition, the change in the path lengths of the jaw wires compensates for the movement of the jaw wires on the side of the end tool due to the pitch motion, and as a result, the pitch motion may be independently performed.

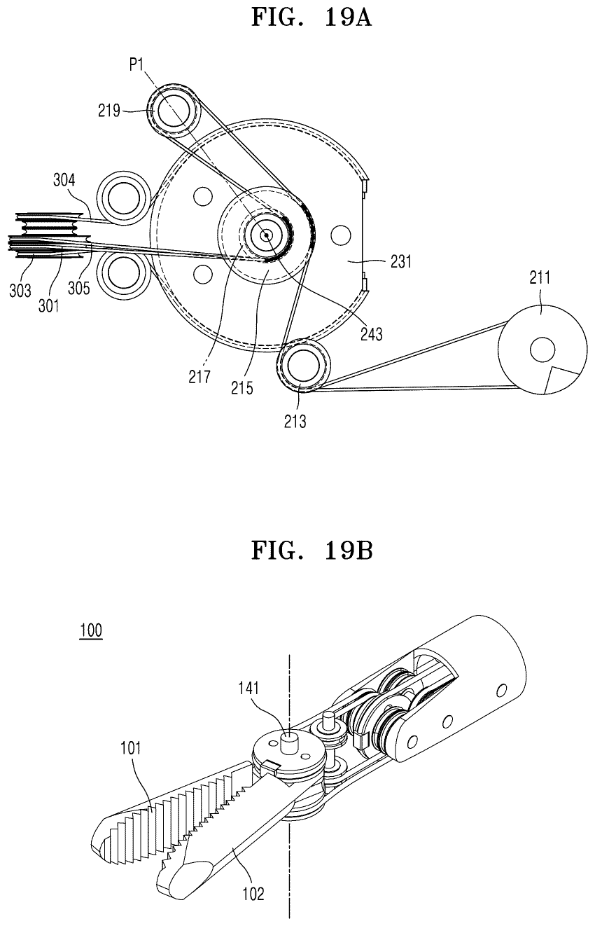

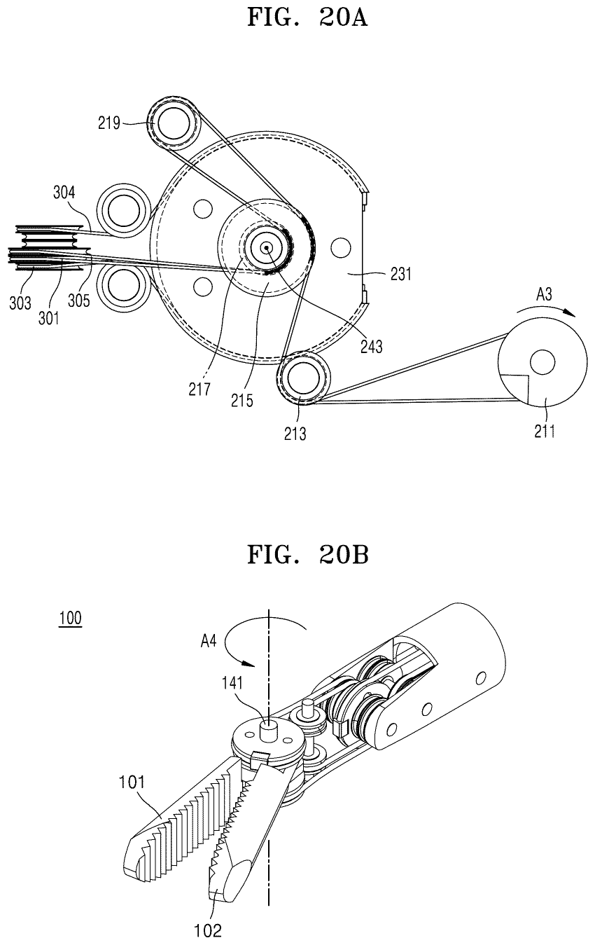

FIGS. 19 and 20 are diagrams illustrating a yaw motion of the multi-joint surgical instrument illustrated in FIG. 6.

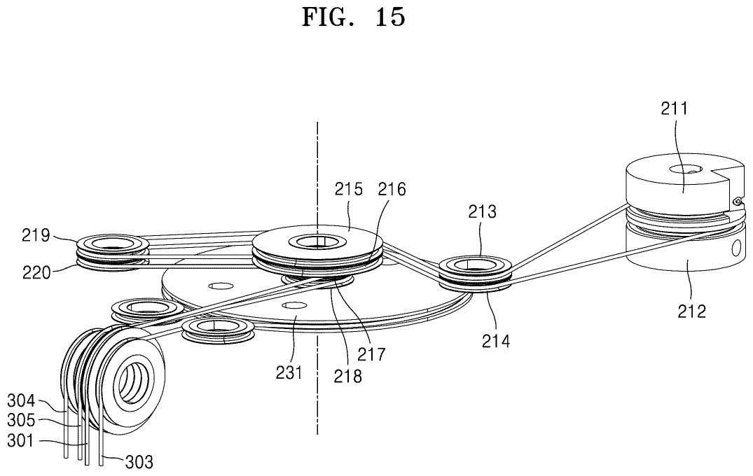

Referring to FIGS. 15, 16, 19, 20 and the like, when the pulley 211, which is a driving part first jaw pulley, rotates in the direction of the arrow A3 for a yaw motion, any one of the wire 301 and the wire 305, which are first jaw wires, is wound around the pulley 211 and the other one is unwound from the pulley 211, due to the rotation of the pulley 211. Accordingly, as the pulley 111, which is an end tool first jaw pulley connected to the opposite side of the wire 301 and the wire 305, rotates in the direction of the arrow A4, the yaw motion is performed.

At this time, the pulley 219, the pulley 220, the pulley 229, and the pulley 230, which are driving part satellite pulleys, and the pulley 215, the pulley 216, the pulley 217, the pulley 218, the pulley 225, the pulley 226, the pulley 227, and the pulley 228, which are driving part relay pulleys, are not changed in position, but only the motion in which the wire 301 and the wire 305 are wound around or unwound from the driving part satellite pulley and the driving part relay pulley occurs.

Accordingly, the driving part pitch pulley rigidly connected to the driving part satellite pulley does not rotate, and the wire 303 and the wire 304, which are pitch wires, are not wound or unwound and maintain their positions.

Similarly, when the pulley 221, which is a driving part second jaw pulley, rotates for a yaw motion, any one of the wire 302 and the wire 306, which are second jaw wires, is wound around the pulley 221 and the other one is unwound from the pulley 221, due to the rotation of the pulley 221. Accordingly, as the pulley 121, which is an end tool second jaw pulley connected to the opposite side of the wire 302 and the wire 306, rotates in any one direction, the yaw motion is performed.

At this time, the pulley 219, the pulley 220, the pulley 229, and the pulley 230, which are driving part satellite pulleys, and the pulley 215, the pulley 216, the pulley 217, the pulley 218, the pulley 225, the pulley 226, the pulley 227, and the pulley 228, which are driving part relay pulleys, are not changed in position, but only the motion in which the wire 302 and the wire 306 are wound around or unwound from the driving part satellite pulley and the driving part relay pulley occurs.

Accordingly, the driving part pitch pulley rigidly connected to the driving part satellite pulley does not rotate, and the wire 303 and the wire 304, which are pitch wires, are not wound or unwound and maintain their positions.

Accordingly, even when the pulley 211 or the pulley 221, which is a driving part jaw pulley, rotates for a yaw motion or an actuation motion, the overall length of the wire 301, the wire 302, the wire 305, and the wire 306, which are jaw wires, within the driving part 200 remains constant.

As described above, in the multi-joint surgical instrument 30 according to an embodiment of the present disclosure, when the driving part pitch pulley rotates, the driving part satellite pulley revolves around the rotation shaft of the driving part pitch pulley to change the path length of the jaw wire wound around the driving part relay pulley, and thus, the jaw wire is wound or unwound in response to the rotation of the driving part pitch pulley, such that the movement of the jaw wire due to the pitch drive may be offset or compensated for, and as a result, the effect of separating the pitch motion and the yaw motion from each other may be obtained.

However, as described above, the pitch motion and the yaw motion are not limited to being mechanically separated from each other, and may be independently separated from each other by a processor of the present disclosure according to an embodiment, and thus able to perform each of the pitch motion and the yaw motion.

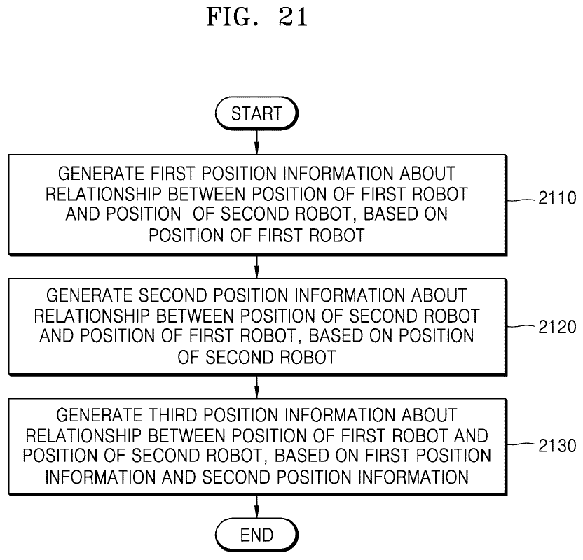

FIG. 21 is a flowchart for describing an example of a method of driving a surgical instrument, according to an embodiment.

Referring to FIG. 21, in operation 2110, the processor 2011 generates first position information about a relationship between the position of a first robot and the position of a second robot, based on the position of the first robot. For example, the processor 2011 may generate first position information including relative position information about the position of the second robot with respect to the position of the first robot. As another example, the processor 2011 may generate first position information including an angle value between a line connecting the position of the first robot to the position of the second robot, and a first axis that is set based on the position of the first robot.

The first position information may include position information about a relationship between the position of the first robot and the position of the second robot, which is generated based on the position of the first robot. For example, the first position information may include relative position information about the position of the second robot with respect to the position of the first robot. As another example, the first position information may include an angle value between a line connecting the position of the first robot to the position of the second robot, and a first axis that is set based on the position of the first robot.

The first axis may include an axis that is set with respect to a certain direction of the first robot. For example, the first axis may include an axis that is set to be perpendicular to a plane facing a forward direction of the first robot. Here, the forward direction may refer to, but is not limited to, the direction in which robotic arms of the first robot perform surgical motions.

In operation 2120, the processor 2011 generates second position information about a relationship between the position of the second robot and the position of the first robot, based on the position of the second robot. For example, the processor 2011 may generate second position information including relative position information about the position of the first robot with respect to the position of the second robot. As another example, the processor 2011 may generate second position information including an angle value between a line connecting the position of the second robot to the position of the first robot, and a second axis that is set based on the position of the second robot.

The second position information may include position information about a relationship between the position of the second robot and the position of the first robot, which is generated based on the position of the second robot. For example, the second position information may include relative position information about the position of the first robot with respect to the position of the second robot. As another example, the second position information may include an angle value between a line connecting the position of the second robot to the position of the first robot, and a second axis that is set based on the position of the second robot.

The second axis may include an axis that is set with respect to a certain direction of the second robot. For example, the second axis may include an axis that is set to be perpendicular to a plane facing a forward direction of the second robot. Here, the forward direction may refer to, but is not limited to, the direction in which robotic arms of the second robot perform surgical motions.

In operation 2130, the processor 2011 generates third position information about a relationship between the position of the first robot and the position of the second robot, based on the first position information and the second position information. For example, the processor 2011 may generate third position information including a relative angle generated based on the position of the first robot and the position of the second robot.

The third position information may refer to position information generated based on each of the position of the first robot and the position of the second robot. For example, the third position information may include a relative angle generated based on the first axis including the position of the first robot and the second axis including the position of the second robot.

In addition, the processor 2011 may generate driving information based on the third position information. In addition, the processor 2011 may control at least one of the first robot and the second robot based on the driving information. For example, the processor 2011 may control a surgical instrument mounted on the second robot, based on the driving information generated based on the third position information. Here, the direction of a motion of the surgical instrument may be identical to the direction of a motion of the surgical instrument shown in an image captured by a camera mounted on the first robot.

$${\vec{p}}_{sTool} = {R}_{sTool \to sRobot} {R}_{sRobot \to cRobot} {R}_{cRobot \to cView} {\vec{p}}_{master} \qquad [ Equation 1 ]$$

Referring to Equation 1, the processor 2011 may calculate driving information \vec{p}_{sTool} based on motion information \vec{p}ₘₐₛₜₑᵣ about a master robot, transformation information R_{cRobot→cView} between coordinates in an image captured by the camera and coordinates of the first robot, transformation information R_{sRobot→cRobot} between the position of the first robot and the position of the second robot, and transformation information R_{sTool→sRobot} between the position of the surgical instrument and the position of the second robot.

The motion information \vec{p}ₘₐₛₜₑᵣ about the master robot may include motion information about the master robot generated by a manipulation by a user, and may be identical to motion information \vec{p}_{cView} about the surgical instrument captured by a camera mounted on the first robot.

The transformation information R_{cRobot→cView} between the coordinates in the image captured by the camera and the coordinates of the first robot may include a transformation matrix between the coordinates in the image captured by the camera and the coordinates of the first robot. The transformation information R_{cRobot→cView} between the coordinates in the image captured by the camera and the coordinates of the first robot may be determined by mechanical information about each of the first robot and the second robot (e.g., the position of the first robot and the position of the second robot).

The transformation information R_{sRobot→cRobot} between the position of the first robot and the position of the second robot may include a transformation matrix between the position of the first robot and the position of the second robot. Here, the transformation information R_{sRobot→cRobot} between the position of the first robot and the position of the second robot may be determined based on a relative angle. In other words, the transformation information R_{sRobot→cRobot} between the position of the first robot and the position of the second robot may be determined by a relative angle that is generated based on a first axis including the position of the first robot, and a second axis including the position of the second robot.

The transformation information R_{sTool→cRobot} between the position of the surgical instrument and the position of the second robot may include a transformation matrix between the position of the surgical instrument and the position of the second robot. The transformation information R_{sTool→cRobot} between the position of the surgical instrument and the position of the second robot may be determined by mechanical information about each of the first robot and the second robot (e.g., the position of the first robot and the position of the second robot).

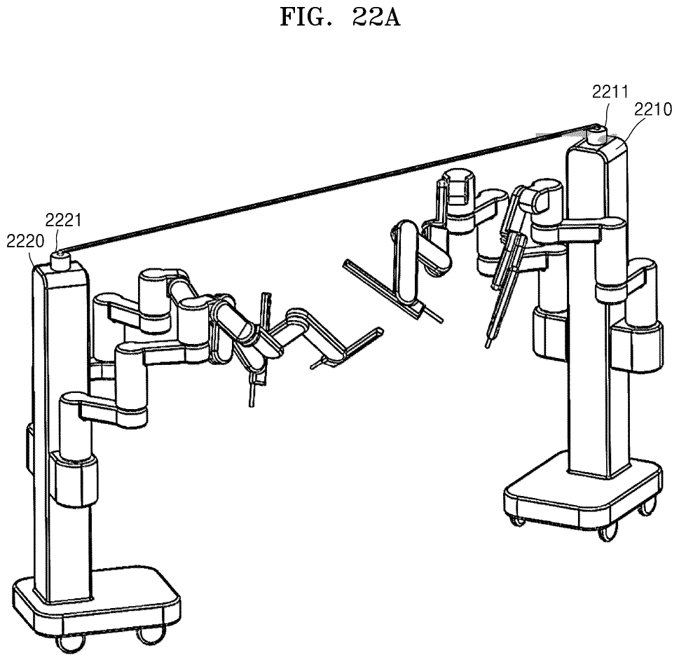

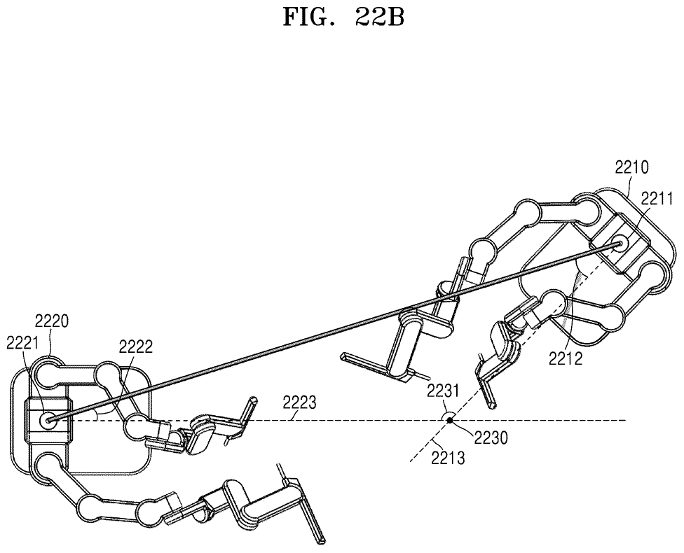

FIGS. 22A and 22B are diagrams for describing examples of a first robot and a second robot, according to an embodiment.

Each of a first robot 2210 and a second robot 2220 may be a slave robot whose motions are controlled by a master robot. The first robot 2210 may be a robot equipped with a camera. For example, at least one robotic arm of the first robot 2210 may be equipped with a camera, or at least one end effector mounted on the first robot 2210 may include or be equipped with a camera. The second robot 2220 may be a robot equipped with a surgical instrument. For example, at least one robotic arm of the second robot 2220 may be equipped with a surgical instrument.

In addition, the first robot 2210 may include a first sensor part 2211. The first sensor part 2211 may be a device that is mounted on the first robot 2210 to be rotatable. For example, the first sensor part 2211 may include a laser transmission part and laser reception part, which are mounted on an upper portion of the first robot 2210 to be able to rotate 360 degrees. Similarly, the second robot 2220 may include a second sensor part 2221. The second sensor part 2221 may be a device that is mounted on the second robot 2220 to be rotatable. For example, the second sensor part 2221 may include a laser transmission part and laser reception part, which are mounted on an upper portion of the second robot 2220 to be able to rotate 360 degrees. However, examples of the first sensor part 2211 and the second sensor part 2221 are not limited thereto.

The first robot 2210 may generate first position information based on the first sensor part 2211. For example, the first sensor part 2211 mounted on the first robot 2210 may sense the position of the second sensor part 2221 through a motion such as rotation. For example, the processor 2011 may calculate an angle value 2212 between a first axis 2213 and a line connecting the position of the first sensor part 2211 and the position of the second sensor part 2221 that is sensed by the first sensor part 2211. The angle value 2212 may be included in the first position information.

The second robot 2220 may generate second position information based on the second sensor part 2221. For example, the second sensor part 2221 mounted on the second robot 2220 may sense the position of the first sensor part 2211 through a motion such as rotation. For example, the processor 2011 may calculate an angle value 2222 between a second axis 2223 and a line connecting the position of the second sensor part 2221 and the position of the first sensor part 2211 that is sensed by the second sensor part 2221. The angle value 2222 may be included in the second position information.

The processor 2011 may generate third position information about a relationship between the position of the first robot and the position of the second robot, based on the first position information and the second position information. For example, based on the angle value 2212 included in the first position information and the angle value 2222 included in the second position information, the processor 2011 may calculate a relative angle 2231 that is generated based on the position of the first robot and the position of the second robot. In detail, the angle value 2212 included in the first position information, the angle value 2222 included in the second position information, and the relative angle 2231 may have a correlation that their sum total is 180 degrees. The relative angle 2231 may include an angle value generated based on a center point 2230 at which the first axis 2213 and the second axis 2223 intersect. The relative angle 2231 may be included in the third position information.

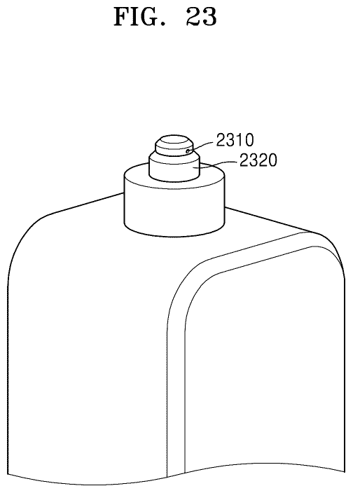

FIG. 23 is a diagram for describing examples of a first sensor part and a second sensor part mounted respectively on the first robot and the second robot illustrated in FIGS. 22A and 22B.

Each of the first sensor part and the second sensor part may include a laser transmission part 2310 and a laser reception part 2320. The laser transmission part 2310 may rotate by a preset angle. For example, the laser transmission part 2310 may rotate 360 degrees. The laser transmission part 2310 may emit a laser that travels in a direction perpendicular to the ground. For example, a laser emitted from the laser transmission part 2310 mounted on the first robot may be detected by the laser reception part mounted on the second robot. In addition, a laser emitted from the laser transmission part mounted on the second robot may be detected by the laser reception part 2320 mounted on the first robot. For example, the laser reception part 2320 may detect a laser emitted from the laser transmission part mounted on the second robot, by using a camera. When the laser reception part 2320 mounted on the first robot detects a laser, the laser transmission part mounted on the second robot may stop emitting a laser.

For example, the laser transmission part of the first robot may rotate while emitting a laser that travels in a direction perpendicular to the ground. In addition, the laser reception part of the second robot may detect the laser emitted from the first robot. When the laser reception part of the second robot detects a laser, the laser transmission part of the first robot may stop emitting a laser and rotating. The processor 2011 may obtain the angle by which the laser transmission part of the first robot has rotated. In this case, the angle by which the laser transmission part of the first robot has rotated, which is obtained by the processor 2011, may be included in the first position information.

In the above-described example, it has been described that each of the first sensor part and the second sensor part includes a laser transmission part and a laser reception part, but the present disclosure is not limited thereto. For example, each of the first sensor part and the second sensor part may include an infrared transmission part and an infrared reception part, and otherwise, the first sensor part and the second sensor part may include means for detecting each other.

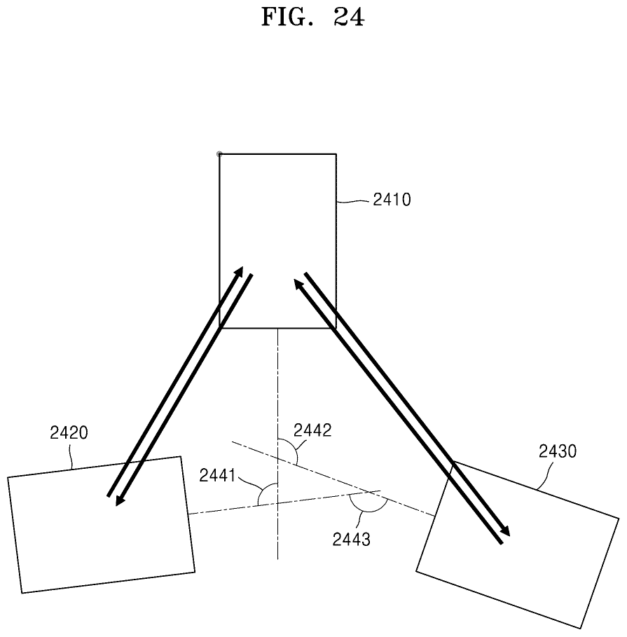

FIG. 24 is a diagram for describing an example in which a processor generates fifth position information, according to an embodiment.

Referring to FIG. 24, the processor 2011 may generate first position information about a relationship between the position of a first robot 2410 and the position of a second robot 2420, based on the position of the first robot 2410, generate second position information about a relationship between the position of the second robot 2420 and the position of the first robot 2410, based on the position of the second robot 2420, and generate third position information 2441 about a relationship between the position of the first robot 2410 and the position of the second robot 2420, based on the first position information and the second position information.

In addition, the processor 2011 may generate fourth position information about a relationship between the position of the first robot 2410 and the position of a third robot 2430, based on the position of the first robot 2410, generate fifth position information about a relationship between the position of the third robot 2430 and the position of the first robot 2410, based on the position of the third robot 2430, and generate sixth position information 2442 about a relationship between the position of the first robot 2410 and the position of the third robot 2430, based on the fourth position information and the fifth position information.

In addition, the processor 2011 may generate seventh position information 2443 about a relationship between the position of the second robot 2420 and the position of the third robot 2430 based on the third position information 2441 and the sixth position information 2442.

For example, the sum of an angle value included in the third position information 2441, an angle value included in the sixth position information 2442, and an angle value included in the seventh position information 2443 may be 360 degrees. Based on a correlation between the angle value included in the third position information 2441, the angle value included in the sixth position information 2442, and the angle value included in the seventh position information 2443, the processor 2011 may use only two values among the angle value included in the third position information 2441, the angle value included in the sixth position information 2442, and the angle value included in the seventh position information 2443, to calculate the other angle value.

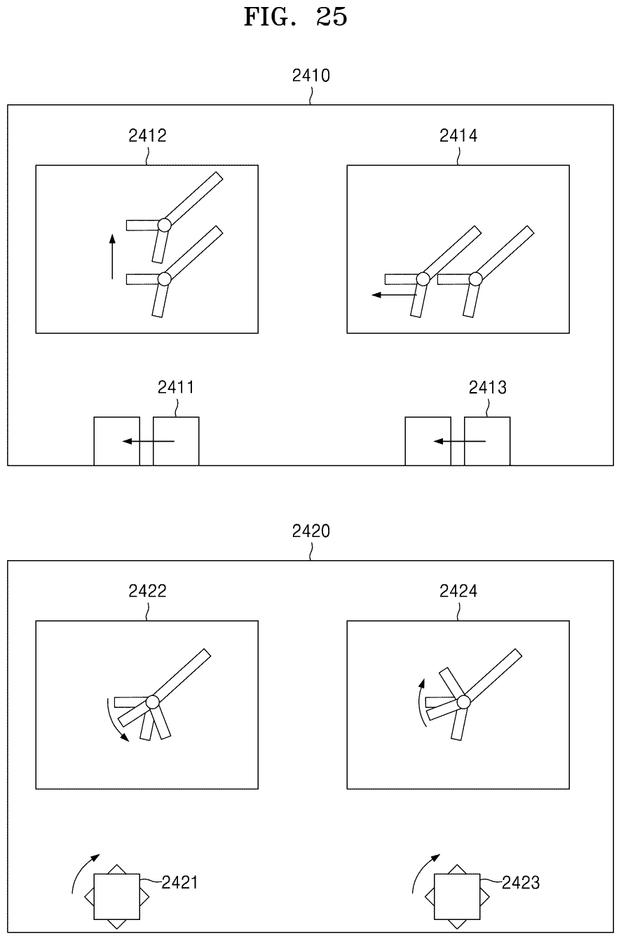

FIG. 25 is a diagram for describing an example of a surgical instrument that operates based on driving information, according to an embodiment.

Referring to FIG. 25, display parts 2412, 2414, 2422, and 2424 may display images captured by a camera mounted on a first robot. The camera mounted on the first robot may capture an image of a surgical instrument mounted on a second robot.

First, in relation to a manipulation 2410 for moving the surgical instrument, when a means 2411 for allowing a user to control the position and function of a surgical instrument is moved to the left, but the movement of the surgical instrument shown on the display part 2412 is in a vertical direction, it becomes difficult for the user to intuitively manipulate the surgical instrument.

On the contrary, in relation to the manipulation 2410 for moving the surgical instrument, when a means 2414 for allowing a user to control the position and function of a surgical instrument is moved to the left, the above-described surgical instrument operating based on driving information is displayed on the display part 2414 as moving to the left. This allows the user to perform surgery with the surgical robot by more accurately reflecting intuitive manipulation by the user.

In addition, in relation to a manipulation 2420 for moving the surgical instrument, when a means 2421 for allowing a user to control the position and function of a surgical instrument is moved clockwise, but the movement of the surgical instrument shown on the display part 2422 is in the counterclockwise direction, it becomes difficult for the user to intuitively manipulate the surgical instrument.

On the contrary, in relation to the manipulation 2420 for moving the surgical instrument, when a means 2423 for allowing a user to control the position and function of a surgical instrument is moved clockwise, the above-described surgical instrument operating based on driving information is displayed on the display part 2414 as moving clockwise. This allows the user to perform surgery with the surgical robot by more accurately reflecting intuitive manipulation by the user.

As described above, the processor 2011 may reflect a manipulation by a user in a motion of a surgical robot more accurately and intuitively, by generating first position information about a relationship between a position of a first robot and a position of a second robot, based on the position of the first robot, generating second position information about a relationship between the position of the second robot and the position of the first robot, based on the position of the second robot, and generating third position information about a relationship between the position of the first robot and the position of the second robot, based on the first position information and the second position information.

According to an embodiment of the present disclosure, a manipulation by a user may be reflected in a motion of a surgical robot more accurately and intuitively, by generating first position information about a relationship between a position of a first robot and a position of a second robot, based on the position of the first robot, generating second position information about a relationship between the position of the second robot and the position of the first robot, based on the position of the second robot, and generating third position information about a relationship between the position of the first robot and the position of the second robot, based on the first position information and the second position information.

In addition, in the present disclosure, a manipulation by the user may be reflected in motions of a plurality of robotic arms included in each of a plurality of slave robots.

Effects of the present disclosure are not limited to the foregoing, and other unmentioned effects would be clearly understood by those skilled in the art from the following description.

Meanwhile, the above-described method may be written as a computer-executable program, and may be implemented in a general-purpose digital computer that executes the program by using a computer-readable recording medium. In addition, the structure of the data used in the above-described method may be recorded in a computer-readable recording medium through various units. The computer-readable recording medium includes a storage medium, such as a magnetic storage medium (e.g., ROM, RAM, a universal serial bus (USB) drive, a floppy disk, or a hard disk) and an optically readable medium (e.g., a CD-ROM or a DVD).

The above-described method may be provided in a computer program product. The computer program product may be traded as commodities between sellers and buyers. The computer program product may be distributed in the form of a machine-readable storage medium (e.g., a CD-ROM), or may be distributed online (e.g., downloaded or uploaded) through an application store (e.g., Play Store™) or directly between two user devices. In a case of online distribution, at least a portion of the computer program product may be temporarily stored in a machine-readable storage medium such as a manufacturer's server, an application store's server, or a memory of a relay server.

It will be understood by those of skill in the art that the present disclosure may be implemented in a modified form without departing from the intrinsic characteristics of the descriptions provided above. Therefore, the disclosed methods should be considered in an illustrative rather than a restrictive sense, and the scope of the present disclosure should be defined by claims rather than the foregoing description, and should be construed to include all differences within the scope equivalent thereto.

What is claimed is:

1. A method comprising:
generating first position information about a relationship between a position of a first robot and a position of a second robot, based on the position of the first robot;
generating second position information about a relationship between the position of the second robot and the position of the first robot, based on the position of the second robot; and
generating third position information about a relationship between the position of the first robot and the position of the second robot, based on the first position information and the second position information.

2. The method of claim 1, wherein the first position information comprises relative position information about the position of the second robot with respect to the position of the first robot, and
the second position information comprises relative position information about the position of the first robot with respect to the position of the second robot.

3. The method of claim 1, wherein the first position information comprises an angle value between a line connecting the position of the first robot to the position of the second robot, and a first axis that is set based on the position of the first robot, and
the second position information comprises an angle value between a line connecting the position of the second robot to the position of the first robot, and a second axis that is set based on the position of the second robot.

4. The method of claim 1, wherein the generating of the first position information comprises generating the first position information based on a first sensor part that is mounted on the first robot to be rotatable, and
the generating of the second position information comprises generating the second position information based on a second sensor part that is mounted on the second robot to be rotatable.

5. The method of claim 1, wherein the third position information comprises a relative angle generated based on the position of the first robot and the position of the second robot.

6. The method of claim 1, further comprising:
generating fourth position information about a relationship between the position of the first robot and a position of a third robot, based on the position of the first robot;
generating fifth position information about a relationship between the position of the third robot and the position of the first robot, based on the position of the third robot;
generating sixth position information about a relationship between the position of the first robot and the position of the third robot, based on the fourth position information and the fifth position information; and
generating seventh position information about a relationship between the position of the second robot and the position of the third robot, based on the third position information and the sixth position information.

7. The method of claim 1, further comprising controlling at least one of the first robot and the second robot, based on the third position information.

8. The method of claim 7, wherein the controlling comprises:
generating driving information based on a motion of a master robot controlling the first robot and the second robot, and transformation relationships between the position of the first robot equipped with a camera, the position of the second robot equipped with a surgical instrument, and a position of the surgical instrument; and
controlling at least one of the first robot and the second robot, based on the driving information.

9. The method of claim 8, wherein the generating of the driving information comprises:
generating first intermediate driving information based on a transformation relationship between a position in an image captured by the camera and the position of the first robot, and motion information about the master robot;
generating second intermediate driving information based on a transformation relationship between the position of the first robot and the position of the second robot, and the first intermediate driving information; and
generating the driving information based on a transformation relationship between the position of the surgical instrument and the position of the second robot, and the second intermediate driving information.

10. A device comprising:
at least one memory; and
at least one processor;
wherein the at least one processor configured to generate first position information about a relationship between a position of a first robot and a position of a second robot, based on the position of the first robot, generate second position information about a relationship between the position of the second robot and the position of the first robot, based on the position of the second robot, and generate third position information about a relationship between the position of the first robot and the position of the second robot, based on the first position information and the second position information.

11. The device of claim 10, wherein the first position information comprises relative position information about the position of the second robot with respect to the position of the first robot, and
the second position information comprises relative position information about the position of the first robot with respect to the position of the second robot.

12. The device of claim 10, wherein the first position information comprises an angle value between a line connecting the position of the first robot to the position of the second robot, and a first axis that is set based on the position of the first robot, and
the second position information comprises an angle value between a line connecting the position of the second robot to the position of the first robot, and a second axis that is set based on the position of the second robot.

13. The device of claim 10, wherein the at least one processor further configured to generate the first position information based on a first sensor part that is mounted on the first robot to be rotatable, and generate the second position information based on a second sensor part that is mounted on the second robot to be rotatable.

14. The device of claim 10, wherein the third position information comprises a relative angle generated based on the position of the first robot and the position of the second robot.

15. The device of claim 10, wherein the at least one processor is further configured to generate fourth position information about a relationship between the position of the first robot and a position of a third robot, based on the position of the first robot, generate fifth position information about a relationship between the position of the third robot and the position of the first robot, based on the position of the third robot, generate sixth position information about a relationship between the position of the first robot and the position of the third robot, based on the fourth position information and the fifth position information, and generate seventh position information about a relationship between the position of the second robot and the position of the third robot, based on the third position information and the sixth position information.

16. The device of claim 10, wherein the at least one processor is further configured to control at least one of the first robot and the second robot, based on the third position information.

17. The device of claim 16, wherein the at least one processor is further configured to generate driving information based on a motion of a master robot controlling the first robot and the second robot, and transformation relationships between the position of the first robot equipped with a camera, the position of the second robot equipped with a surgical instrument, and a position of the surgical instrument, and control at least one of the first robot and the second robot, based on the driving information.

18. The device of claim 17, wherein the at least one processor is further configured to generate first intermediate driving information based on a transformation relationship between a position in an image captured by the camera and the position of the first robot, and motion information about the master robot, generate second intermediate driving information based on a transformation relationship between the position of the first robot and the position of the second robot, and the first intermediate driving information, and generate the driving information based on a transformation relationship between the position of the surgical instrument and the position of the second robot, and the second intermediate driving information.

19. A computer-readable recording medium having recorded thereon a program for causing a computer to execute the method of claim 1.
